(19) United States
(12) Patent Application Publication
(10) Pub. No.: US 2018/0228462 A1
(43) Pub. Date: Aug. 16, 2018

(54) WEARABLE ULTRASOUND DEVICE

(71) Applicant: UltraSense Medical Inc., San Jose, CA (US)

(72) Inventors: Mo Maghsoudnia, San Jose, CA (US); Carl Herickhoff, Los Altos, CA (US)

(73) Assignee: UltraSense Medical Inc., San Jose, CA (US)

(21) Appl. No.: 15/891,301

(22) Filed: Feb. 7, 2018

Related U.S. Application Data
(60) Provisional application No. 62/455,763, filed on Feb. 7, 2017.

(51) Int. Cl. A61B 8/08; G06T 7/62; A61B 8/00; B06B 1/02; B06B 1/06; H01L 41/04; H01L 41/047; H01L 41/08; H01L 41/25; G01F 22/00; G01N 29/24

(52) U.S. Cl. CPC A61B 8/08; A61B 5/746; A61B 8/5223; A61B 8/4236; B06B 1/0215; B06B 1/0622; H01L 41/042; H01L 41/0475; H01L 41/0825; H01L 41/25; G01F 22/00; G01N 29/2437; G06T 2207/10132; G06T 2207/30076; G06T 7/62

(57) ABSTRACT

Systems, methods, and apparatuses for non-invasively measuring volumes of a body cavity by a wearable ultrasonic device are provided. The wearable ultrasonic device can collect data related to the body cavity perform diagnostic measurements associated with the body cavity to provide alerts to user. The wearable ultrasonic device can be affixed to the skin of a user's body. The wearable ultrasonic device can include an ultrasonic transducer that transmits and receives ultrasonic signals directed toward the body cavity such as a user's urinary bladder. The device can include processing electronics coupled to the ultrasonic transducer to process the ultrasonic signals to determine the volume of the body cavity and transmit alerts to the user when the volume reaches a particular threshold. The processing electronics can be integrated with the ultrasonic transducer to operate at low voltages and be battery powered.

### CROSS-REFERENCES TO RELATED APPLICATIONS

This application is a non-provisional application claiming priority to U.S. Provisional Patent Application No. 62/455,763 entitled “Wearable Ultrasound Device” filed on Feb. 7, 2017.

### FIELD

The present disclosure relates generally to systems and methods of monitoring internal organs using an ultrasonic wearable device, and more particularly to wearable ultrasound devices for non-invasively measuring the volume of fluid in a body cavity such as a urinary bladder. Methods and systems used for a wearable ultrasonic transducer are also described.

### BACKGROUND

Urinary incontinence affects millions of people all over the world. Such incontinence causes discomfort and embarrassment, sometimes to the point of social isolation. Generally, incontinence occurs when bladder sphincter muscles are not capable of retaining urine within the bladder as bladder pressure and distension increases. Urinary incontinence often results, for example, from weakened pelvic muscles that are unable to suppress the flow of urine when a common occurrence such as coughing, laughing, or mild physical exertion causes an increase in abdominal (and bladder) pressure. In many cases, incontinence consistently occurs when the bladder volume reaches a maximum volume threshold for a given individual. Additionally, millions of children suffer from nocturnal enuresis. Nocturnal enuresis results in the involuntary loss of urine during sleep in children 0-12 years old. Children with nocturnal enuresis are more likely to have behavioral disorders and low self-esteem than their non-wetting peers. Also, diabetic autonomic neuropathy causes the nerves of the bladder to no longer respond to pressure as the bladder fills with urine. Diabetic autonomic neuropathy can lead to increased risk of urinary tract infections and involuntary loss of urine. Thus, real-time non-invasive measurement of bladder volume and notification of approaching an individual's particular threshold volume may help to prevent incontinence and treat nocturnal enuresis.

While conventional image-based ultrasound devices can be used to measure bladder volume, such devices are large, expensive, require extensive electronics, and have high operating voltages, such that they are not sufficiently mobile for everyday use outside of a hospital or clinical setting. Further, conventional devices require a device operator to manually define a cavity area on a two-dimensional image, from which the conventional ultrasound device can determine a cross-section measurement of the defined area. Because of the conventional device size and voltage limitations and the requirements for input from a trained operator, bladder measurements are typically only performed periodically in a hospital or clinical setting. Moreover, because conventional devices are used in hospital or clinical settings, they often do not include connectivity needed to notify users of monitored parameters.

Accordingly, wearable ultrasound devices that address the foregoing problems are desirable.

### SUMMARY

Embodiments provide systems, methods, and apparatuses for non-invasively measuring volumes of fluids in a body cavity by a wearable ultrasonic device are provided. The wearable ultrasonic device may collect data related to the body cavity and provide a user alerts to improve the management a variety of bladder ailments such as diabetic autonomic neuropathy, nocturnal enuresis, and urinary incontinence. For example, a portable ultrasound device can be affixed to the skin of an user's body. An ultrasonic transducer incorporated in the device may transmit and receive ultrasonic signals directed towards a body cavity such as a user's urinary bladder. The device may include processing electronics coupled to the ultrasonic transducer to process the ultrasonic signals to determine the volume of the body cavity. If the body cavity is the user's urinary bladder, the device may determine the volume of fluid in the urinary bladder in real time and may generate alerts when the volume reaches a particular threshold so that the individual can manage evacuation and prevent incontinence. The wearable device with processing electronics coupled to the ultrasonic transducer can operate at low voltages and, as a result, be battery powered.

In some embodiments, the device may non-invasively measure a volume of a body cavity as the volume of the body varies over time. For example, the processing electronics may be configured to receive reflected signals associated with emitted ultrasonic signals reflected from a target region associated with the wearable device. The processing electronics may digitize the reflected signals. In some embodiments, the processing electronics may transmit the digital reflected signals to measurement circuitry to analyze the signal information, and determine the volume of the fluid in the body cavity based on the received reflected signals. The processing electronics may be further configured to store digital reflected signals, compare the determined volume of the fluid with a threshold volume and output an alert when the determined volume reaches or exceeds the threshold volume. The threshold volume may be user-specific, and the alert may include a haptic alert generated by an actuator, an audible alert generated by a speaker or a light emitting diode coupled to the processing electronics. By determining the volume of fluid in the body cavity such as the urinary bladder and/or alerting the user as described above, the device may help the user or a supervising medical professional manage voiding of the urinary bladder and avoid incontinence.

According to other embodiments, a method of measuring a volume of a body cavity is provided. The volume of the body cavity may be determined using an ultrasonic signal emitted from an array of transducers. The ultrasonic signal can be emitted along different lines through the body cavity in a target region. The reflections from the front wall of the body cavity (e.g., a bladder) may be measured first, and reflections from the back wall measured second. The measurements for each line can provide a pair a peaks: one peak for the echo from the front side of the body cavity and another peak for the echo from the backside of the body cavity. The volume of the body cavity can be determined using a time difference between the respective peaks for each of a plurality of lines. The peaks may be identified in specified time windows, which may be determined during a calibration phase using machine learning to identify a best fit for the times associated with the first peak and the second peak when the body cavity is at different volumes (e.g., empty and full). Thus, different time windows of different models may be used to search for the peaks corresponding to different lines. Additional channels may be used, each potentially having a delay between received signals from a same point on the body cavity.

The wearable electronic device may include transducer circuitry coupled to processing circuitry in a combined device. The transducer circuitry may include one or more piezoelectric micromachined ultrasonic transducers (PMUTs). The transducer circuitry may be provided on a first substrate and the processing circuitry may be formed on a device wafer. One or more of cavities may be formed on the device wafer to isolate the processing circuitry from ultrasonic signals generated by the transducer circuitry. The combined device may be formed by bonding a device oxide layer of the processing circuitry to a bonding oxide layer of the transducer circuitry. The combined device may be further processed to form vias and contacts coupled the processing circuitry and transducer circuitry. The combined device may operate at lower voltages than existing ultrasonic devices.

A better understanding of the nature and advantages of embodiments of the present invention may be gained with reference to the following detailed description and the accompanying drawings. In the drawings, like reference numerals represent like parts throughout similar views.

### BRIEF DESCRIPTION OF THE DRAWINGS

FIG. 1 shows an illustration of wearable device positioned on a user's body for non-invasively measuring a volume of a fluid in a body cavity according to an embodiment of the present invention.

FIG. 2 shows an exploded view of a wearable device for non-invasively measuring a volume of a fluid in a body cavity according to an embodiment of the present invention.

FIG. 3A shows an array of piezoelectric micromachined ultrasonic transducers used in a wearable device as described above according to an embodiment of the present invention.

FIG. 3B shows a simplified illustration of a two-dimensional array of piezoelectric micromachined ultrasonic transducers used in a wearable device according to an embodiment of the present invention.

FIG. 4 shows a cross-sectional view of a single piezoelectric micromachined ultrasonic transducer bonded to a CMOS wafer used in a wearable device according to an embodiment of the present invention.

FIG. 5A shows a block diagram of the processing electronics board according to an embodiment of the present invention.

FIG. 5B shows a simplified illustration of a receiving coil configured to receive wireless energy according to an embodiment of the invention.

FIG. 5C shows a simplified elevation view of the processing electronics board according to an embodiment of the present invention.

FIG. 6 shows a simplified schematic of a wearable device in communication with other devices according to an embodiment of the present invention.

FIG. 7 shows a device for volume measurement and monitoring of a body cavity according to an embodiment of the present invention.

FIG. 8 shows a flowchart illustrating the operation of a wearable ultrasonic device according to an embodiment of the present invention.

FIG. 9 shows a simplified block diagram of a PMUT/ASIC stack according to an embodiment of the present invention.

FIG. 10A shows a simplified diagram of processing the signal received on a plurality of channels according to an embodiment of the present invention.

FIGS. 10B and 10C illustrate return echoes associated with various sized body cavities disposed in the range of coverage of a channel according to an embodiment of the present invention.

FIG. 11 shows a cross-sectional view of a single piezoelectric micromachined ultrasonic transducer bonded to a CMOS wafer used in an ultrasonic wearable device according to an embodiment of the present invention.

FIGS. 12A-12H illustrate a device stack corresponding to process steps used to fabricate piezoelectric micromachined ultrasonic transducers bonded to an application specific integrated circuit according to an embodiment of the present invention.

FIG. 13 shows a block diagram of an example computer system usable with system and methods according to embodiments of the present invention.

### DETAILED DESCRIPTION

In contrast with conventional ultrasound devices, methods and systems for a wearable ultrasonic device are provided. As examples, wearable devices can be worn on the body, such as affixed to a user's skin (potentially with an intervening membrane) or as part of material used in clothing. A wearable device may maintain approximate contact with a user's body, e.g., throughout daily activities. Wearables can include devices that collect data using various electronic circuits and sensors to generate data related a user's health. In addition to portability, wearable devices described herein can determine body cavity information without requiring inputs from a trained operator, e.g., just the user. Also, wearable devices described herein may integrate transducer and signal processing circuitry to reduce the necessary transducer bias voltage.

A wearable ultrasonic device can include a coupling hydrogel pad for affixing the device to a surface of the body and providing acoustic coupling interface between the device and the body. The device can include at least one ultrasonic transducer coupled to the coupling pad interface and configured to emit ultrasonic signals directed towards a target region and receive returning ultrasonic echo signals associated with the emitted ultrasonic signals. The device can further include processing electronics coupled to at least one ultrasonic transducer and a battery coupled to and configured to power at least one ultrasonic transducer and the processing electronics.

In many embodiments, the device may determine parameters associated with a cavity of the body. For example, the device may non-invasively measure a volume of a fluid in a body cavity. In some embodiments, the processing electronics may determine the parameters associated with the body cavity. For example, the processing electronics may be configured to store information associated with the returning signals, analyze the signal information, and determine the volume of the fluid in the body cavity based on the stored information. In particular, the body cavity may be a cavity in a urinary bladder. The processing electronics may thus be configured to determine a volume of a fluid in the urinary bladder. The processing electronics may be further configured to compare the determined volume of the fluid with a threshold volume and output an alert when the determined volume reaches or exceeds the threshold volume. The threshold volume may be user-specific, and the alert may include a haptic alert generated by an actuator coupled to the processing electronics. By determining the volume of fluid in the body cavity such as the urinary bladder and/or alerting the user as described above, the device may help the user or a supervising medical professional manage voiding of the urinary bladder and avoid incontinence.

The device may also be helpful to identify irregularities in voiding. For example, the processing electronics may be configured to determine the volume of a fluid in the urinary bladder after voiding by the user. The processing electronics may be further configured to identify voiding dysfunction based on the determined volume after voiding. By tracking the determined volume after voiding and/or comparing it to normal values, the user, a medical professional, or the device's processing electronics itself may be able to identify irregular or dysfunctional voiding so that it can be addressed and treated appropriately.

The device may also be helpful to determine other parameters, in addition to volume of fluid in the body cavity. For example, the processing electronics may be configured to determine a volume of the structure surrounding the body cavity, such as the total urinary bladder volume (i.e. the capacity of the bladder), based on the stored information associated with the returning signals. Since the total urinary bladder volume may be different for an individual, this may help to determine an appropriate user-specific threshold volume of fluid therein that warrants notification or alerting of the user. As another example, the processing electronics may be configured to determine a thickness of a wall of the urinary bladder. The thickness of the wall may also vary for individuals, and may also aid in determining the appropriate user-specific threshold volume.

In many embodiments, the device may further include a wireless radio coupled to the processing electronics. For example, the wireless radio may be configured to transmit stored information associated with the return signals to a remote computing device. The remote computing device may thus be configured to determine the volume of the fluid in the body cavity based on the stored information. Determination of the volume or other parameters by a remote computing device may help to reduce the size, complexity, and power requirements of the processing electronics. As another example, the processing electronics may be configured to transmit alerts or other stored information as described above to a remote device (such as a smart phone) via the wireless radio. The processing electronics may also be configured to receive information from a remote device via the wireless radio. For example, the processing electronics may be configured to receive control signals configured to calibrate the device and/or modify alerts via the wireless radio. As another example, the processing electronics may be configured to receive user-specific data from a remote device via the wireless radio. Such user-specific data may help in determining the parameters and/or threshold values described above.

In many embodiments, the battery may be a rechargeable battery so that the device may be used on a prolonged basis. In some cases, the device may include receiving coils that are electrically coupled to the rechargeable battery. The receiving coils may be configured to receive energy form an external energy source wirelessly and transmit the energy to recharge the rechargeable battery. For example, the receiving coils may be configured to receive energy from an external energy source via radio frequency transmission.

The coupling interface may also aid in transmission of ultrasonic signals. For example, the coupling interface may include an adhesive hydrogel layer configured to adhere to the skin surface of a user. The hydrogel may thus act as a conductive medium that improves the bond between the skin and the transducers, thus ensuring more efficient transmission of the ultrasonic signals between the body and the transducers.

The device may be designed to minimize the size and required operating voltage while maintaining the required functionality. To achieve such size and operability, at least one ultrasonic transducer may include an array of piezoelectric micromachined ultrasonic transducers (PMUT). The array of PMUTs may be formed on a microelectromechanical system (MEMS) wafer that is bonded to a CMOS wafer containing the processing electronics. The interconnect between the CMOS and the PMUTs may be a thru-silicon-via (TSV). In some embodiments, each PMUT may include an active piezoelectric layer including aluminum nitride, a passive elastic layer including single-crystal Silicon, and top and bottom electrodes including Molybdenum. The active piezoelectric layer may include scandium doped aluminum nitride. In some embodiments, the array of PMUTs may be configured to have a driving voltage of less than 4 Volts. For example, the array may be configured to generate a maximum acoustic intensity of up to 80 mW/cm2 at a depth of up to 3 cm with an AC input driving voltage of 3.3 Volts. The array may be configured to have a frequency response of 800 KHz to 1.0 MHz.

The device's size may be minimized to allow for easy and non-invasive wearing by the patient. For example, the device may have a cross section that measures 2 cm by 4 cm and may have a thickness of 0.5 cm or less. This way, the device may be easily secured to a user's skin and may be easily hidden from view by the user's clothing without losing functionality.

The arrangement of the PMUTs on the device may allow for capturing particular features of the user's body. In many embodiments, the PMUTs may be spaced apart a predetermined distance so as to form an aperture that encompasses a particular body cavity for which information is desired. For example, the PMUTs may be spaced apart a distance so that a resulting aperture encompasses the urinary bladder of a user. In some cases, the PMUTs may be spaced apart roughly 5-6 mm.

In addition to identifying tissue structures and characteristics thereof, the device may be used therapeutically. In many embodiments, the array of PMUTs may be configured to emit ultrasonic signals that release therapeutic compositions within a targeted region of the user's body. For example, the PMUTs may be used to non-invasively rupture microbubbles containing therapeutic compositions when the microbubbles are in a targeted region.

### I. Wearable Device

Aspects of the present disclosure relate to wearable devices for non-invasively measuring volumes of fluids in a body cavity. In particular, a portable wearable ultrasound device may be provided that can be affixed to the skin of an user's body. An array of ultrasonic transducers incorporated in the device may transmit and receive ultrasonic signals directed towards a body cavity such as the user's urinary bladder. The device may process the ultrasonic signals to determine the volume of fluid in the urinary bladder in real time and may generate alerts when the volume reaches a particular threshold so that the individual can manage evacuation and prevent incontinence.

A. Example Placement of Wearable Device

Embodiments of a wearable ultrasonic device are provided herein. The wearable ultrasonic device may be positioned by a user to continuously monitor a portion of the user's body. In some embodiments, the user may position the wearable ultrasonic device to monitor bladder function. The wearable ultrasonic device may emit ultrasonic pulses receive echoes to collect data related to bladder function. The collected data may be processed to provide the user bladder information and alerts to assist with managing a variety of bladder issues such as diabetic autonomic neuropathy, nocturnal enuresis, and urinary incontinence. The collected data, bladder information, and alerts may be transmitted to a user's mobile device, to an electronic device of a treating physician, and/or a cloud data server for further analysis and alerts.

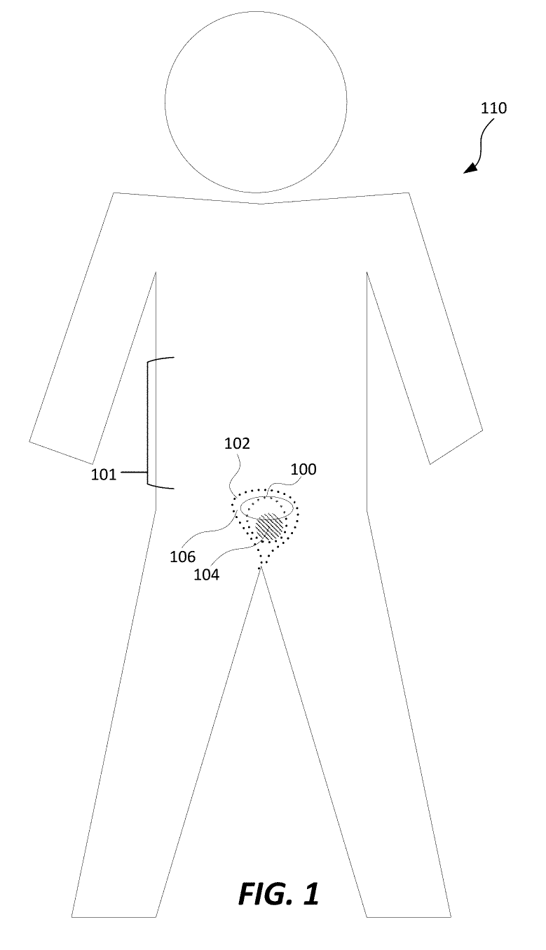

FIG. 1 shows an illustration of wearable device positioned on a user's body for non-invasively measuring a volume of a fluid in a body cavity according to an embodiment of the present invention. As shown in FIG. 1 a user 110 may affix a wearable device 100 under the abdomen 101 in order to position the transducers above so that the sub-apertures capture the user's bladder 102. As described below, device 100 may be adhered directly to the user's skin using an adhesive coupling interface (not shown in FIG. 1), which may only require applying the adhesive coupling interface to the user's skin with moderate force. Once it is secured in place, device 100 may be operable to analyze various features of the user's bladder 102 to aid in managing urinary incontinence. This may include, but is not limited to, the volume of fluid 104 disposed in bladder 102 at any given time, the thickness of the bladder wall 106, the shape of the bladder 102, any other feature of bladder 102, and/or any combination thereof. In other embodiments, the device 100 may be positioned to monitor another area of the user 110. Device 100 may be configured to monitor another body cavity or function such as the heart, vascular system, and the like.

B. Example Layers of Wearable Device

The wearable device may be constructed of various layers. Each layer may provide different functionality. As described above, the wearable device can be attached to the skin of a person. One layer can help with such attachment, or such a coupling layer can be considered as an addition to the wearable device, as opposed to an integrated part.

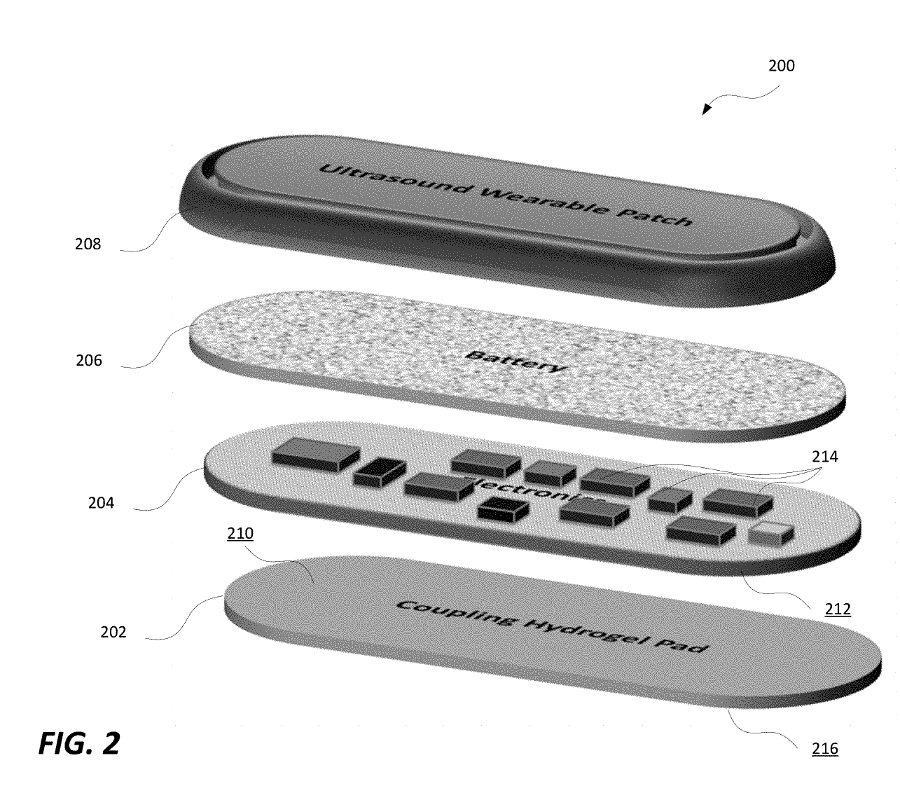

FIG. 2 shows an exploded view of the wearable device 200 for non-invasively measuring a volume of a fluid in a body cavity according to an embodiment of the present invention. Device 200 (e.g. 100 from FIG. 1) may include a coupling interface 202, a processing electronics board 204, a battery 206, and a cover 208. As will be described in further detail below, device 200 may include one or more ultrasonic transducers (not shown in detail in FIG. 2) that enable it to insonify and thus interrogate a volume of tissue in a user's body, such as for the purpose of non-invasively measuring a volume of fluid in a body cavity, such as a urinary bladder. In particular, the ultrasonic transducers are integrated into the processing electronics board 204. While any suitable ultrasound techniques may be used to obtain the requisite information for determining the volume, in some embodiments, the transducers may direct ultrasonic signals towards the body cavity (such as the bladder) at a fundamental frequency, receive returning echoes of the ultrasonic signals, convert these echoes into digital form and store this digital information in memory on the device.

The transducers may be coupled to and/or integrated with the processing electronics board 204, which may have additional processing electronics 214 that are configured to control the transducers, receive information from the transducers, process the information to determine the volume, store the information in memory, and/or transmit any of the information to a remote device for further processing or output as will be described in further detail below. In some embodiments, the processing electronics 214 may be implemented using field programmable gate array. Battery 206 may be electrically coupled to the processing electronics board 204 to power the processing electronics 214 and the ultrasonic transducers. In some embodiments, battery 206 may be a rechargeable battery. In other embodiments, battery 206 can integrated with the processing electronics board 204. For convenient charging, although not shown in FIG. 2, device 200 may include a charging spiral that allows the battery to be recharged inductively. Alternatively, battery 206 may be removed for charging and/or replacement.

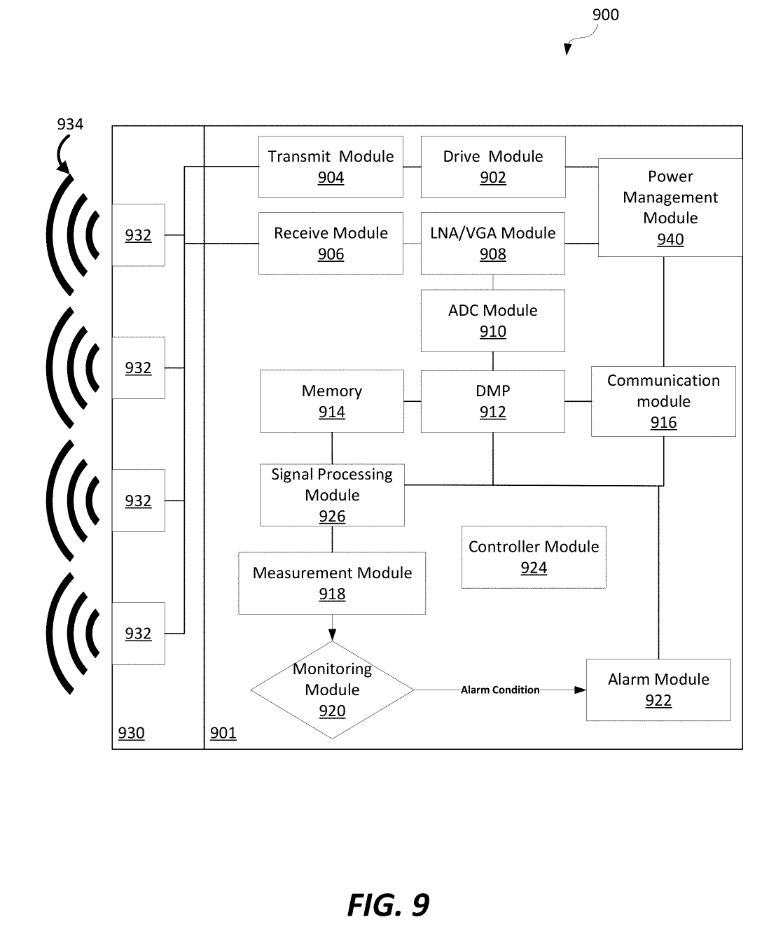

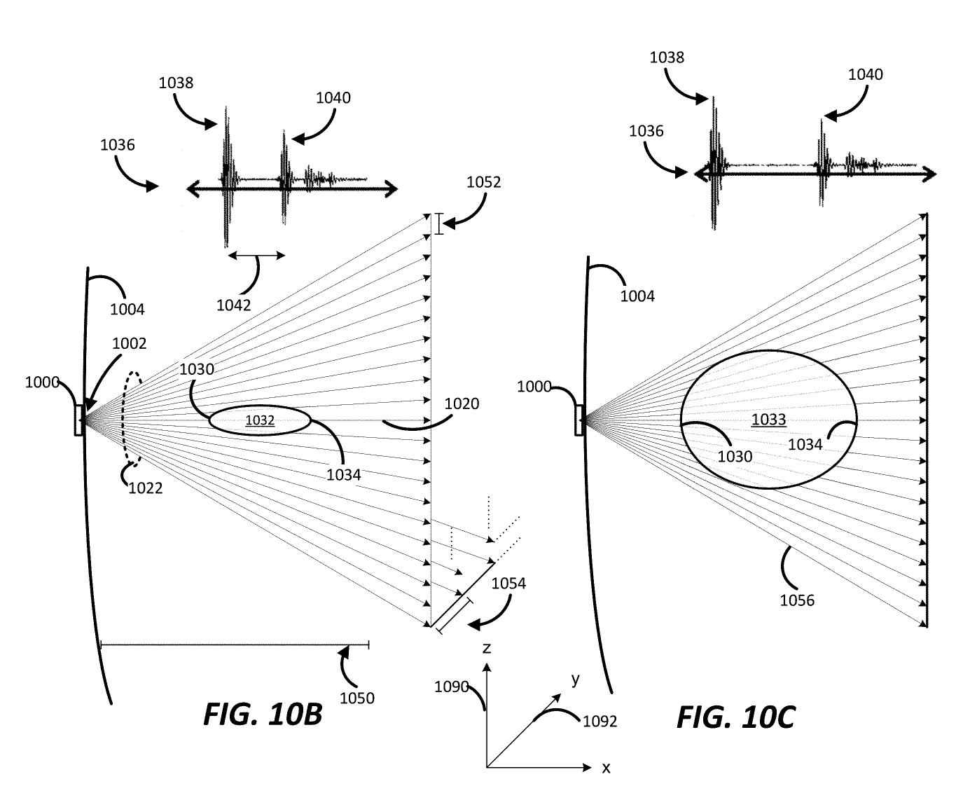

Although shown in an exploded view in FIG. 2, processing electronics board 204, battery 206, and cover 208 may be assembled to form a single assembly for use in operation. In order to allow convenient replacement of any of coupling interface 202, processing electronics board 204, battery 206, and cover 208, some or all of these components may be removably coupled to one another by any suitable means. For example, coupling interface 202 may have an adhesive on a top surface 210 that interfaces with bottom surface 212 of the processing electronics 214 which incorporate the ultrasonic transducers (as will be described in further detail with respect to FIGS. 7 and 9 below). The adhesive may allow coupling interface 202 to remain affixed to processing electronics 214 under normal operation, but also allow removal of coupling interface 202 upon a moderate pulling force by a user. This may allow a user to replace coupling interface 202 as it becomes less effective and/or worn out. Similarly, battery 206 may be removably coupled to processing electronics by any suitable mechanism, including a snap fit connection, adhesive, or any other removable coupling. This may allow battery 206 to be removed so that it can be charged and/or replaced with another battery. In other embodiments, the battery 206 may be non-removably fixed to the processing electronics board 204 to reduce device size and complexity. Finally, cover 208 may be removably coupled to the remaining portion of device by any suitable mechanism, such as a snap fit mechanical connection, an adhesive, or any other removable coupling. For example, cover 208 may be snap fit to the perimeter surface of processing electronics board 204 so as to protect the battery 206 and processing electronics 214 mounted on processing electronics board 204 in operation while allowing access to said components when needed.

Coupling interface 202 may be configured to affix device 200 to a surface of a user's body while also acting as a conductive medium that improves the bond between the skin and the ultrasonic transducers (described in further detail below), thus ensuring more efficient transmission of ultrasonic signals between the body and the transducers. For example, coupling interface 202 may be an adhesive hydrogel pad that improves the bond between a user's skin and the ultrasonic transducers of device 200 that are coupled to processing electronics board 204. As described above, coupling interface 202 may be removably coupled to device 200 via top surface 210 so that it can be replaced when worn out or otherwise as desired by the user. Similarly, bottom surface 216 of coupling interface 202 may include an adhesive layer or adhesive portions so that it can adhere to a user's skin at a desired location for several hours to keep the device in place during daily activity, but still be removed from a user's skin without causing excessive discomfort. In some embodiments, coupling interface 202 may include any suitable components and arrangement thereof for both wearable device 200 to the user's body and conducting ultrasonic signals between the body and transducers. For example, coupling interface 202 may include an adhesive portion along its perimeter with an ultrasound hydrogel or other conductive medium housed in its perimeter.

As described above, device 200 may include ultrasonic transducers that may be used to non-invasively measure a volume of a fluid in a body cavity. While conventional ultrasound imaging devices can be used to measure the volume of fluids in body cavities, these devices are not sufficiently portable or wearable. Indeed, because such devices have high operating voltages (sometimes requiring as much as 100 V) and require extensive electronics, they are limited to imaging systems used onsite at medical centers, and cannot be incorporated in a portable, wearable device for real-time monitoring during daily use. The design of the individual ultrasonic transducers, as incorporated in the processing electronics, and the arrangement of the array of transducers allow device 200 to operate on significantly less voltage while still providing the requisite ultrasonic emission and sensing capabilities needed for accurately measuring volumes of fluid in body cavities non-invasively in real time. Specifically, the device 200 may be designed so that the dimensions are approximately 2 cm×4 cm, with a thickness of 0.5 cm or less. This way, the device may be easily wearable without interrupting the daily activity of the user and easily covered by the user's clothing when in operation.

C. Transducer Array and Stack

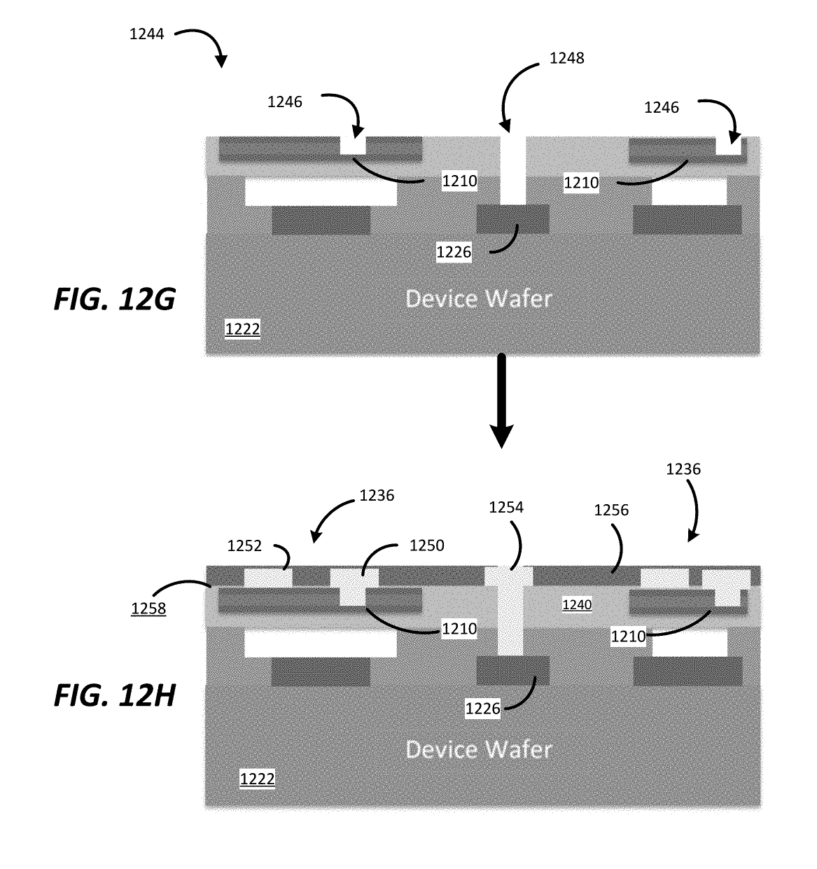

In order to achieve the aforementioned functionality and geometry, device 200 may include an array of piezoelectric micromachined ultrasonic transducers (PMUTs) formed on a microelectromechanical system (MEMS) wafer. The MEMS wafer may be bonded to a complementary metal-oxide-semiconductor (CMOS) wafer, which may include the processing electronics needed to control the PMUT and process the signals received therefrom to monitor the volume of fluid in the relevant body cavity. A process used to form the MEMS wafer will be described below with reference to FIGS. 12A-12H.

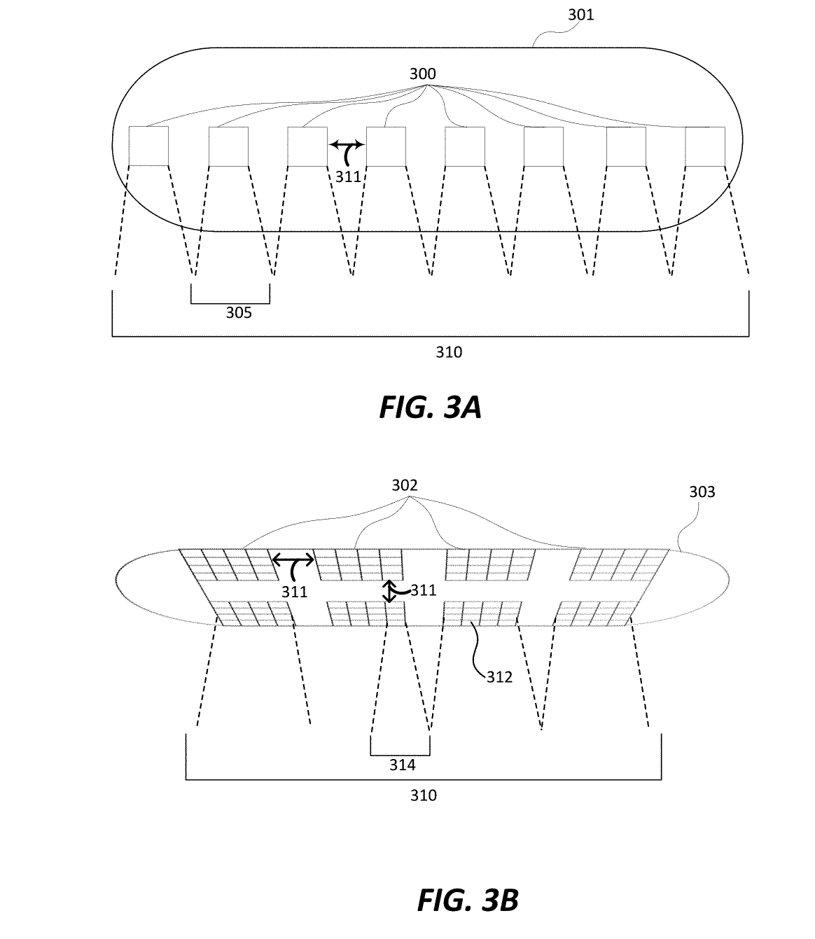

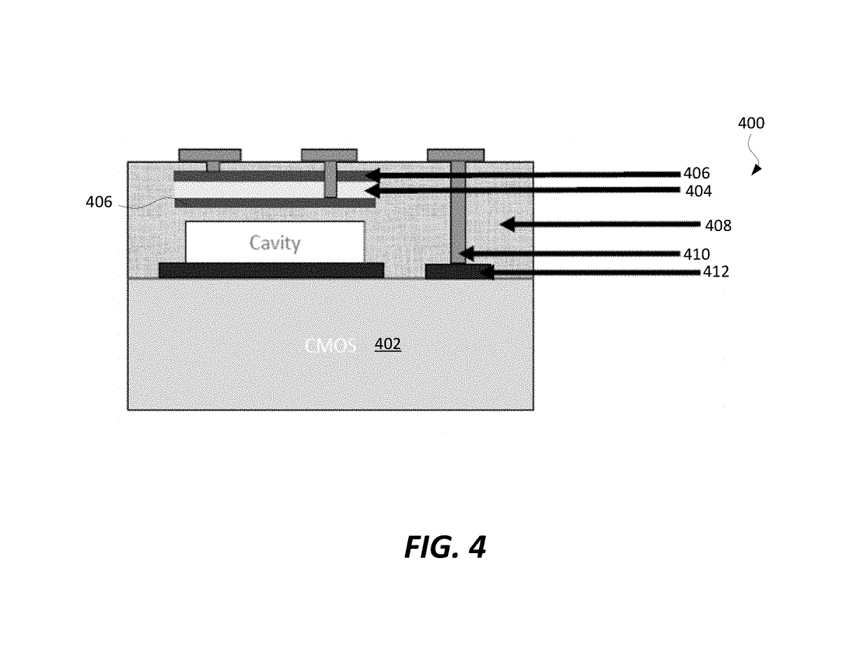

FIG. 3A shows an array of piezoelectric micromachined ultrasonic transducers used in a wearable device as described above, in accordance with many embodiments. As noted above, rather than a single ultrasonic transducer, device 301 may incorporate an array of PMUTs 300 that may operate at a lower voltage than bulk PZT ceramic transducers and capacitive micromachined transducers, and allow device 301 to be portable and wearable. As shown in the simplified view of FIG. 3A, each PMUT 300 may have a range of coverage, or sub-aperture 305, across which it may transmit and receive ultrasonic signals in operation. While these sub-apertures 305 may be smaller than the relevant body cavity to be analyzed, it will be understood that the PMUTs 300 can be arranged so that the information from the set of sub-apertures can be used to effectively generate an aperture 310 to analyze a particular target area. The arrangement of the PMUTs 300 may correspond to a predetermined distance 311 that forms a desired aperture 310. Aperture 310 may form a cone that encompasses the body cavity. For example, if device is used to measure the volume of fluid in a urinary bladder, it may be desirable to have eight PMUTs 300 or “sup aperture tiles” spaced apart approximately 5-6 mm. In some cases, each sub-aperture tile may include 16 PMUT elements, such that each tile generates 16 channels, for a total of 128 channels. This arrangement may provide a suitable aperture with the sufficient number of channels to measure features of a bladder of a user with sufficient resolution. It will be understood that while a linear array of PMUTs 300 is shown in FIG. 3A for illustrative purposes, other suitable arrangements may be used depending on the desired body cavity to be measured.

FIG. 3B shows a simplified illustration of a two-dimensional array of piezoelectric micromachined ultrasonic transducers used in a wearable device 303 according to an embodiment of the present invention. Rather than a single ultrasonic transducer, each sub-aperture tile 302 may include 16 elements 312. The 16 elements 312 may be arranged in an array. For example, sub-aperture tile 302 may be a 4×4 array of the 16 elements 312. Each element 312 may correspond to a channel. In some embodiments, each channel can be made of an array of transducers. In a particular embodiment, each element 312 may include 49 PMUTs. Integrating multiple elements 312 into a single sub-aperture tile 302 can reduce overall size of the transducer array allowing a reduced operating voltage, and facilitating a portable and wearable device 303. As shown in the simplified view of FIG. 3B, each element 312 may have a range of coverage, or sub-aperture 314, across which it may transmit and receive ultrasonic signals in operation. While these sub-apertures 314 may be smaller than the relevant body cavity to be analyzed, it will be understood that each element 312 can be arranged so that the information from the set of sub-apertures 314 can be used to effectively generate an aperture 310 to analyze a particular target area. The aperture size must be large enough to cover the target area.

For example, if device 303 is used to measure the volume of fluid in a urinary bladder, it may be desirable to have eight sub-aperture tiles 302 arranged in a 4×2 rectangular array. Each sub-aperture tile 302 tile can be approximately 7.2 mm by 7.2 mm. Each sub-aperture tile 302 can be spaced apart approximately 3-4 mm. In some cases, the 16 elements 312, for each of the sub-aperture tiles 302 generates 16 channels, for a total of 128 channels. In some embodiments, each element 312 can be 1.8 mm by 1.8 mm. This arrangement may provide a suitable aperture with the sufficient number of channels to measure features of a bladder of a user with sufficient resolution. It will be understood that while a rectangular array of sub-aperture tiles 302 is shown in FIG. 3B for illustrative purposes, other suitable arrangements may be used depending on the desired body cavity to be measured.

In some embodiments, device 303 may include a single-chip containing 8 PMUT sub-aperture tiles with 16 elements each as described above. As will be understood by those skilled in the art, the number of transmit/receive channels of such a device may be tuned to obtain the desired resolution for a given application. For example, it may be desirable, in the case of measuring bladder features as described herein, to provide a single-chip, 8 sub-aperture tiles by 16 elements per tile device with 128 channels. The device may provide 128 emission channels and 128 receiving channels.

The design and arrangement described above may allow device 303 to produce enough acoustic intensity while operating with a significantly lower voltage than traditional ultrasound equipment. It will be understood that this allows the device to meet the size and battery constraints needed for a wearable device. Specifically, a device 303 as described may run on 4 Volts or less (e.g. 3.3 V), and may generate an acoustic intensity of as much as 80 mW/cm² as many as 3 cm below the contact point with a user's skin. This particular intensity and depth may be suitable for monitoring a user's bladder. While described herein primarily for the purpose of monitoring a user's bladder, it will be understood that the principles described herein may apply to monitoring or otherwise measuring features of other tissues as well.

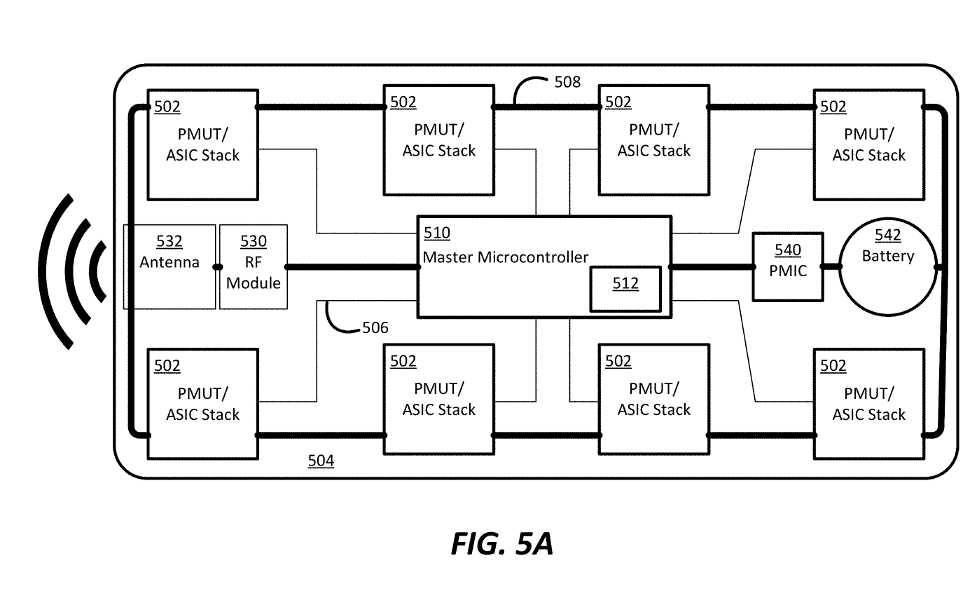

FIG. 4 shows a cross-sectional view of a single piezoelectric micromachined ultrasonic transducer 400 bonded to a CMOS wafer 402 used in a wearable device, as described above, in accordance with many embodiments. As described above, and as shown in FIG. 4, each PMUT 400 may be formed on a MEMS wafer that is bonded to a CMOS wafer 402. In this way, PMUT 400 may be coupled to the requisite processing electronics of the CMOS. It will be understood that each PMUT may have an active piezoelectric layer 404 along with top and bottom electrodes 406. The passive elastic layer 408 may comprise SiO₂ or any other suitable passive layer. The active piezoelectric layer 404 may be approximately 1 μm thick Aluminum Nitride, and the passive elastic layer may be approximately 1 μm thick single-crystal Silicon. In some embodiments, the active piezoelectric layer 404 may be Scandium-doped Aluminum Nitride. Alternatively, the active piezoelectric layer 404 may be another suitable piezoelectric ceramic such as PZT. Both the top and bottom electrodes 406 may comprise Molybdenum. In order to bond the PMUT 400 to the top metal 412 of CMOS wafer 402, fusion bonding via thru-silicon-via (TSV) as shown at 410 may be used. This methodology results in significant parasitic reduction which in turn results in generation of increased acoustic intensity in operation.

In some embodiments, device 303 may include a single-chip containing 8 PMUT sup-aperture tiles with 16 elements each as described above. As will be understood by those skilled in the art, the number of transmit/receive channels of such a device may be tuned to obtain the desired resolution for a given application. For example, it may be desirable, in the case of measuring bladder features as described herein, to provide a single-chip, 8 sub-aperture tiles×16 element PMUT device with 128 channels.

The design and arrangement described above may allow device 100 to produce enough acoustic intensity while operating with a significantly lower voltage than traditional ultrasound equipment. It will be understood that this allows the device to meet the size and battery constraints needed for a wearable device. Specifically, a device 100 as described may run on 4 Volts or less (e.g. 3.3 V), and may generate an acoustic intensity of as much as 80 mW/cm² as many as 3 cm below the contact point with a user's skin. This particular intensity and depth may be suitable for monitoring a user's bladder.

D. Example Circuitry

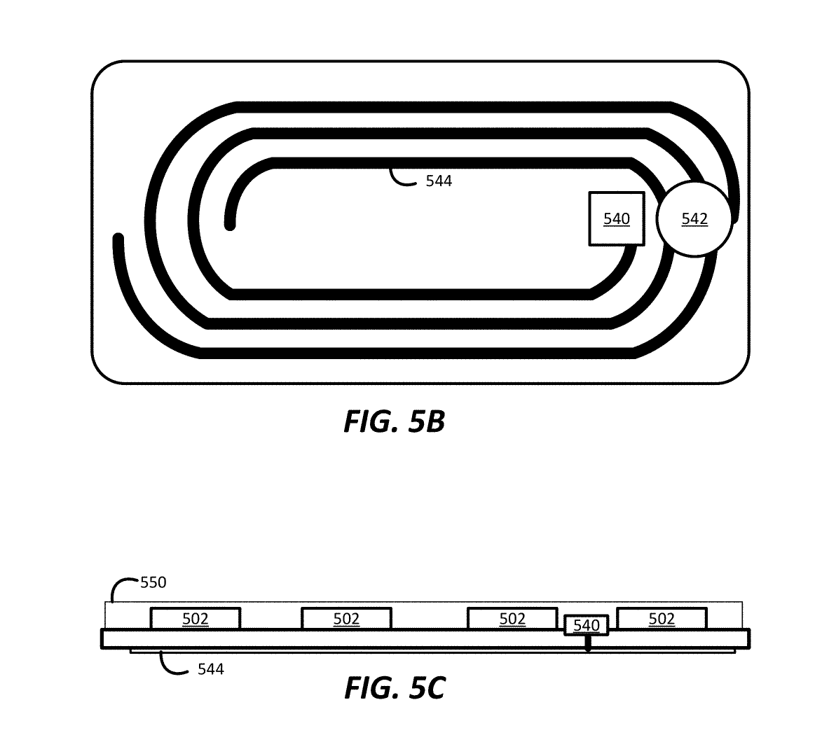

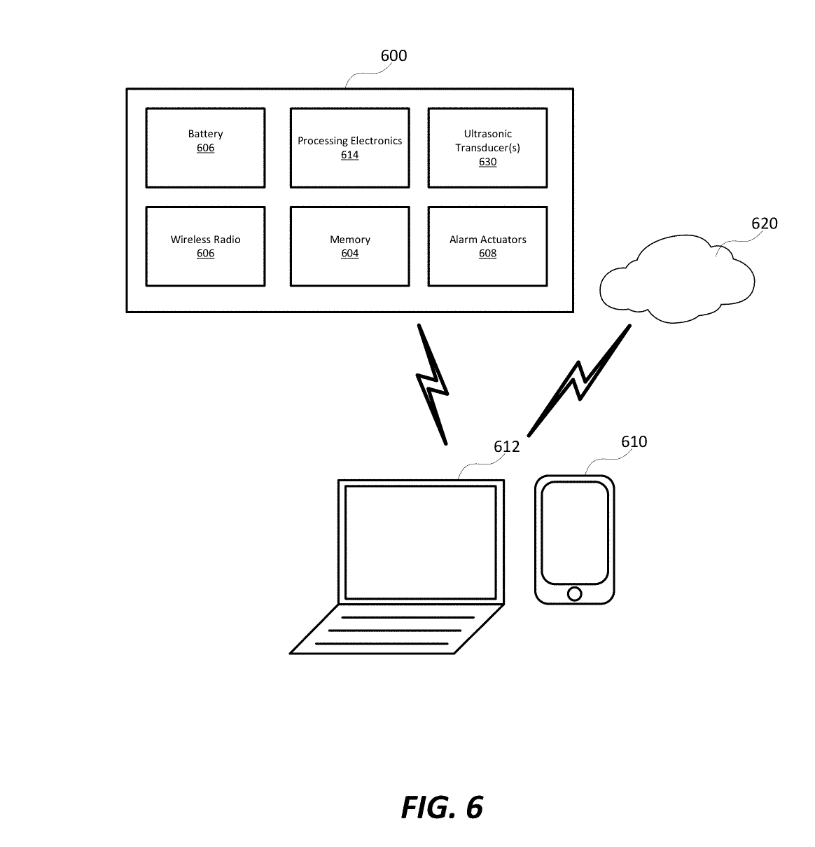

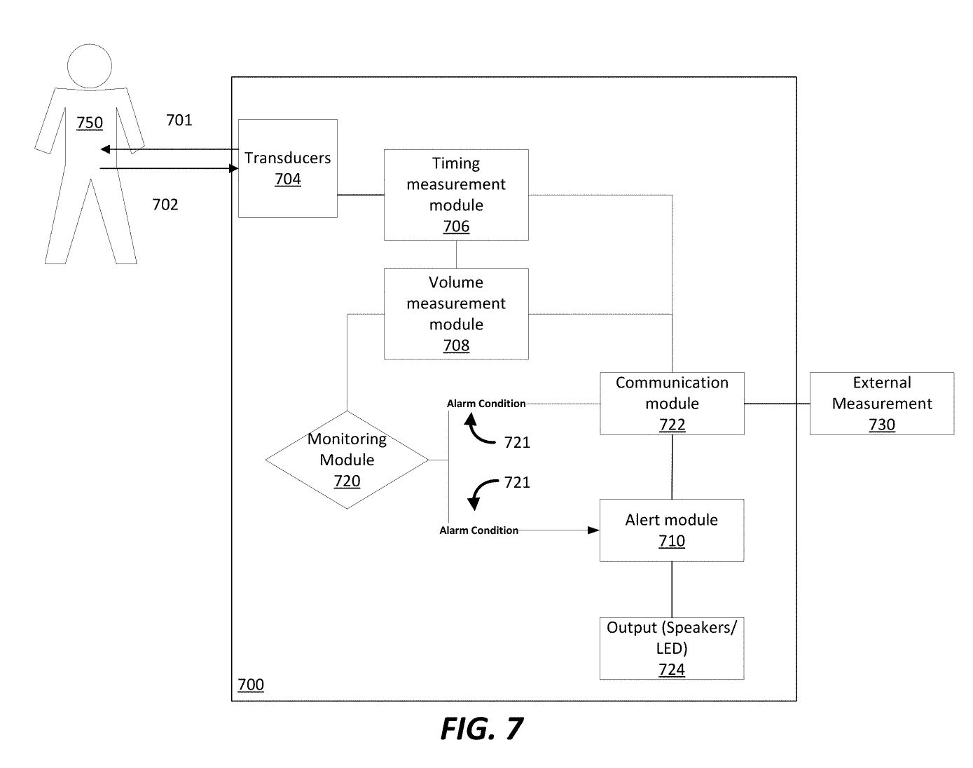

FIGS. 5A-5C show views of the processing electronics board and transducer array. FIG. 5A shows a block diagram of the processing electronics board 204 according to an embodiment of the present invention. The processing electronics board 204 can include PMUT/ASIC integrated stacks 502, a flexible printed circuit board 504, data interconnects 506, power interconnects 508, a master microcontroller 510, a Radio Frequency (RF) module 530, an antenna 532, a battery 542, and a power management integrated circuit (PMIC) 540. In some embodiments, the PMUT/ASIC integrated stacks 502 can comprise a plurality of transducers coupled to an application specific integrated circuit (ASIC). The ASIC can include processing and measurement circuitry. The PMUT/ASIC integrated stacks 502 can include 16 channels per chip. The ASIC can provide multi-channel control with no connection parasitics because the processing and measurement circuitry is coupled directly to the transducer. The ASIC can include voltage drivers to drive the transducers. The processing circuitry can access on-chip memory to perform on-chip beam forming. For the purpose of the description of FIG. 5A, the processing electronics board 204 may be positioned on a user's abdomen to measure the volume of the user's bladder.

In some embodiments, the master microcontroller 510 can be configured to operate the board in a plurality of modes. The plurality of modes includes, standby, startup, transmit, receive, processing. In standby mode, the master microcontroller 510 puts all elements of the processing electronics board 204 in a sleep mode to conserve power. Upon receiving a command or input from the user, the master microcontroller 510 may enter startup mode. In startup mode the master microcontroller may send commands to startup each of the slave ASICs at each PMUT/ASIC integrated stacks 502 and the PMIC 540. The PMIC 540 may generate a voltage to power the devices on the processing electronics board. In some embodiments, the voltage may be approximately 24 V. In transmit mode, the microcontroller may send commands to each of the ASICs coupled to the PMUT/ASIC integrated stacks 502 to fire pulses on all channels. The pulses may be fired at 24 V. After transmitting a pulse, the master microcontroller may send commands to enter receive mode. In receive mode, the transducers in the PMUT/ASIC integrated stacks 502 will generate a signal in response to a receiving echo associated with the previous pulse. As used herein, with reference to the receiving echo, returning signal, reflected signal, and received signal may also be used to describe the receiving echo. The PMUT/ASIC integrated stacks 502 may use processing circuitry in the ASIC to digitize the signal and store the digital reflected signal data in an on-chip memory. In some embodiments, the ASIC includes one or more analog-to-digital converters and an SRAM to store the digital signal data.

The master microcontroller 510 can send a command to enter data processing mode. In this mode, the circuitry in the slave ASICs may be powered off except for memory. The master microcontroller 510 may receive the digital reflected signal data from the on-chip memory of each ASIC. The master microcontroller 510 can further process the digital reflected signal data and perform received data beam-forming. Using the processed data, the master microcontroller 510 may determine a bladder's size. During the data processing mode, the master microcontroller may send commands to the RF module 530 to initiate wireless communication with a remote device. The master microcontroller 510 can send and receive data and commands with the RF module 530. The data can include ultrasound data, digital reflected signal data, bladder size data, and alerts. The RF module 530 may be operable to perform wireless transmission using antenna 532 using a variety of frequencies such as WiFi, Bluetooth, and the like. After transmitting data, the master microcontroller 510 can continuously record data, go into a standby mode and record data at predetermined intervals, or go into a standby mode and record data upon receiving a command from a user.

In some embodiments, the processing electronics may include a beamforming engine. The master microcontroller 510 can include a beamforming module 512 that include circuits, instructions, or the like. The beamforming module 512 may process digital signal data associated with a return echo from one or more of the PMUT/ASIC integrated stacks 502 to reconstruct a far field beam pattern associated with aperture 310. Beamforming module 512 can also focus and steer an ultrasound beam associated with the transducer array, e.g., by providing control signal to PMUT/ASIC integrated stacks 502. When performing a signal analysis, beamforming module 512 may time delay one or more digital signals associated with a return echo according to a delay profile for a target point to focus and steer the beam. The beamforming module 512 may control the directionality, sensitivity, resolution, and signal-to-noise ratio of the system. In some embodiments, the beamforming module can use the digital signal data to determine data associated with 231 beamforming lines that pass through target points.

FIG. 5B shows a simplified illustration of a receiving coil configured to receive wireless energy according to an embodiment of the invention. In some embodiments, the battery 542 may be a rechargeable battery so that the device may be used on a prolonged basis. In some cases, the device may include receiving coils 544 that are electrically coupled to the rechargeable battery 542 and or the PMIC 540. The receiving coils 544 may be configured to receive energy form an external energy source wirelessly and transmit the energy to recharge the rechargeable battery 542. For example, the receiving coils 544 may be configured to receive energy from an external energy source via radio frequency transmission. The receiving coils 544 and PMIC 540 may be configured to support, for example, one of Qi, Power Matters Association, or Association for wireless power standards, and may operate at different frequencies. In some embodiments, the device processing electronics board may include a port for wired charging/wired power.

FIG. 5C shows a simplified elevation view of the processing electronics board according to an embodiments of the present invention. In some embodiments, the processing electronics board 204 may include a cap 550. The cap 550 may provide electrical isolation from the surrounding environment and a smooth surface for the coupling interface 202. The cap 550 may be configured to be transparent to ultrasound. The PMUT/ASIC integrated stacks 502, charging coil 544, and PMIC 540 are also illustrated in FIG. 5C.

E. Communication System Involving Wearable Device

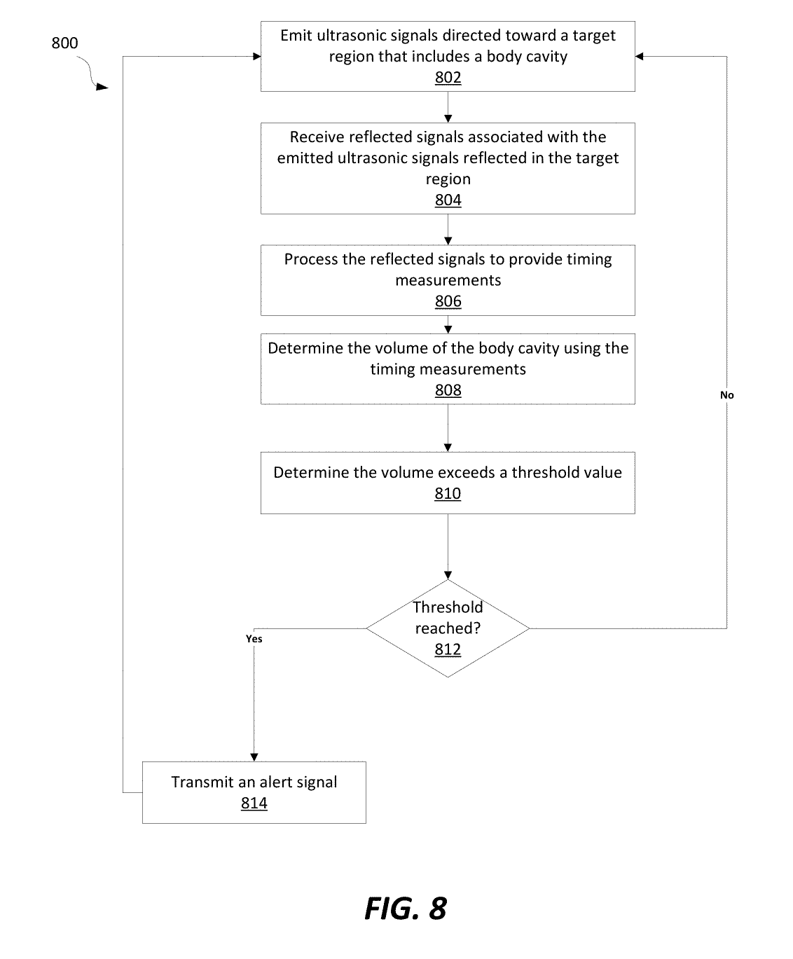

FIG. 6 shows a simplified schematic of a wearable device 600 in communication with other devices, in accordance with many embodiments. In some embodiments, wearable device 600 can be wearable device 100 described in FIGS. 1-5. Wearable device 600 may include a battery that provides power to all of the device components, ultrasonic transducers 630 that send and ultrasonic signals towards the bladder for analysis thereof, and processing electronics 614 which are configured to control the transducers 630, receive the information from the ultrasonic transducers 630, process the information to determine the volume, store the information in memory 604, and/or transmit any of the information to a remote device such as a remote device 610 or a personal computer 612 for further processing or output. In some embodiments, the processing electronics 614 generate digital ultrasound data from the 128 channels at a 10 MHz data rate. The processing electronics 614 may be configured to transmit data using a general purpose serial interface such as a 7-wire interface. Memory 604 may be used to store data received from transducers, patient-specific data used for processing such data, processing algorithms themselves, bladder information calculated using the processing algorithms, bladder information threshold related data, operating parameters for components of device 600, and/or any other data needed for operation of device 600. In some embodiments, the remote device 610 may be any portable mobile electronic device such as a PDA, a smart phone, a tablet, a smartwatch, or the like.

In order to transmit information to a remote device, device 600 may also include a wireless radio 606. Wireless radio 606 may be any suitable radio that can wirelessly transmit information to remote devices, including radios that employ short range, low power communication protocols such as Bluetooth, low power Bluetooth, or other protocols. As further depicted in FIG. 6, information may also be transmitted to and from a data cloud 620 which may include a number of remote servers and/or databases. For example, device 100 may transmit information to the data cloud 620 via remote devices 610 and/or 612, which can be accessed by authorized users and/or authorized third parties such as healthcare providers, drug makers, researchers, and the like. It will be understood that information relating to the bladder monitoring may help to update bladder monitoring algorithms used by device 600, remote devices 610, 612, or data cloud 620 remote servers, may aid healthcare providers and researchers in identifying and diagnosing conditions of a user, and may aid drug makers in assessing whether drugs used while monitoring a user's bladder are effectively treating conditions that cause frequent urination.

As described above, device 600 may be used to alert a patient of conditions related to their bladder, including when the volume of fluid in the bladder reaches a threshold volume. Accordingly, device 600 may include one or more alarm actuators 608. These alarm actuators 608 may provide haptic, visual, and/or audible alarms to the user upon the occurrence of an event, as will be described in further detail below. For example, alarm actuators 608 may vibrate, sound an audible alarm tone, and/or emit a visible light when a user's bladder volume reaches a threshold value. It will be understood that alarm actuators 608 may be configured to provide different notifications for different types of events, including more intense alerts for more serious events. Additionally, it will be understood that alarm actuators 608 may be customized for a particular user to meet his or her specific needs.

### II. Continuous Measurement of Volume and Alerts

Non-invasively measuring and monitoring volume of a fluid in a subject's body cavity (e.g., a urinary bladder) will now be described. As described above, monitoring the volume of fluid in a user's urinary bladder may help the user manage urinary incontinence, nocturnal enuresis, diabetes autonomic neuropathy, and the like. Continuous measurement provided by methods and systems described herein can improve management and treatment of bladder dysfunction compared to conventional devices that offer limited monitoring only in a hospital or clinical setting with a trained operator. Although some examples are described in relation to a bladder, other embodiments can involve other body cavities such as a heart, a kidney, a portion of a vascular system, and the like.

A. Device for Volume Measurement and Alert

FIG. 7 shows a device for volume measurement and monitoring of a body cavity according to an embodiment of the present invention. The device 700 may include one or more ultrasonic transducers 704, a timing measurement module 706, a volume measurement module 708, a monitoring module 720, an alert module 710, a communication module 722, and an output module 724. In some embodiments, communication module 722 may be coupled to an external measurement module 730.

In the example shown, the ultrasonic transducers 704 emit ultrasonic signals 701 toward a body cavity of a user 750. The ultrasonic transducers 704 receive a returning ultrasonic signal 702 associated with the emitted ultrasonic signals 701. The device 700 can convert the returning ultrasonic signals 702 to digital signals and transmit the signals to the timing measurement module 706. In other embodiments, the signals stay analog. The conversion can involve analog-to-digital converters (ADCs) that may be in timing measurement module 706 The timing measurement module 706 may be configured to process the digital signals associated with the returning ultrasonic signal 702. The processing can include aligning digital signals associated with the returning ultrasonic signal, identifying peaks in the digital signal, e.g., by searching specified time windows in the signal. In some embodiments, the timing measurement module 706 may coherently sum the digital signals to form a composite digital signal integrating the digital signals associated with each channel. Device 700 can transmit the timing data to volume measurement module 708 or to the external measurement module 730, via communication module 722, for further processing.

The volume measurement module 708, and/or the external measurement module 730, can determine, based on the received times of the peaks, a roundtrip time for an emitted ultrasonic signal from the first wall to a second wall of a body cavity. Pairs of peaks can be identified as corresponding to a same emission line, where one peak corresponds to a front wall (side) and a second peak corresponds to a back wall (side). A time difference between a pair of peaks can provide the roundtrip time between the front wall and the back wall.

The roundtrip time can be used to determine a volume of the body cavity. The device 700 can route the volume of the body cavity to the monitoring module 720. The monitoring module 720 can determine whether the volume exceeds a threshold value. The threshold value can include a minimum/maximum volume, a rate of change of the volume, and the like. The threshold value may be a percentage of the minimum/maximum volume, and thus the threshold value may be user-specific. The monitoring module 720 can detect an alarm condition 721 based on the threshold value and transmit the alarm condition 721 to one or more of the communication module 722 and an alert module 710. The alert module 710 can include alert circuitry and/or instructions to process the alarm condition 721 and transmit a signal to output module 724 or an external device to trigger an alert for the user. In some embodiments, the alert module 710 and the monitoring module 720 may be combined in a single module. The output module 724 can be at least one or speakers, an LED, a haptic motor, and the like.

B. Method of Performing Emitting Pulses and Performing Measurement

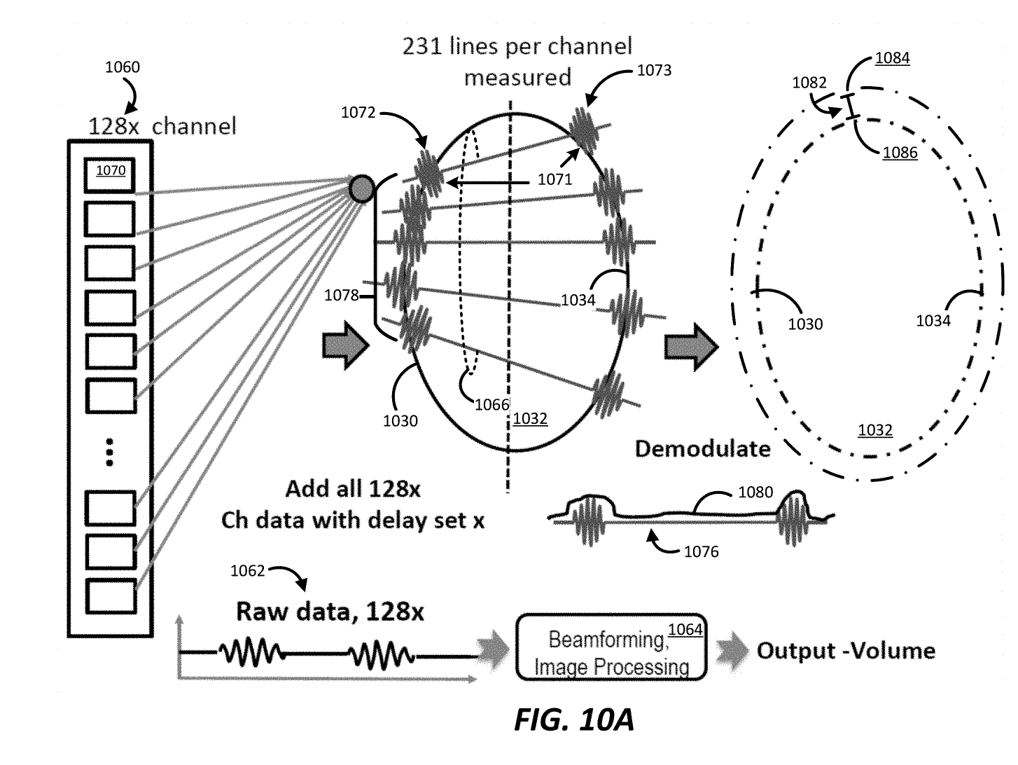

FIG. 8 is a flowchart 800 illustrating the operation of a wearable ultrasonic device, in accordance with many embodiments. It will be understood by those skilled in the art that the order of the steps may be switched, some of the steps may be combined, and/or some of the steps may be optional. The flowchart of FIG. 8 is one example of the method and is not intended to be limiting. Thus, it will be understood by those skilled in the art that various other operation(s) disclosed in this application may be used instead of those shown in FIG. 8. Method 800 may be performed by any or all of the devices and components described above. For example, method 800 may be performed by a wearable device, such as device 100, including transducers processing electronics, associated components thereof and/or any suitable combination thereof. The steps of method 800 will now be described with reference to FIG. 8.

At step 802, a transmit phase may case the transducers of the wearable device to emit ultrasonic signals directed toward a target region that may include a body cavity. In some embodiments, wearable device may transmit ultrasound pulses towards a bladder. As described above, in order to measure a user's bladder volume and related characteristics, the transducers may be configured to generate an acoustic intensity of as much as 80 mW/cm² up to 3 cm below the contact point with a user's skin. As also described above, ultrasound pulses are transmitted towards the bladder at a fundamental frequency. The transmission of the ultrasound pulses may occur for a predetermined period, which period may be referred to as a transmission phase. For example, the transmission phase may last for up to 10 microseconds (i.e. 5 cycles at 0.5 MHz). This may be sufficient to reach the maximum output pressure amplitude from the transducers. Ultrasonic frequencies emitted at step 802 may vary from 20 KHz to 20 MHz depending on a variety of factors such as a wave speed of a fluid associated with the body cavity, desired resolution, the depth and size of a target, and the like.

At step 804, a receive phase, after the transmission phase is completed, may cause the transducers to be configured to receive reflected signals associated with the emitted ultrasonic signals reflected in the target region. Configuring the transducers may include entering a receiving phase, during which returning echoes of the transmitted pulses are received by the transducers. In some cases, there may be a predetermined delay period between the transmission phase and the receiving phase to allow time for the signals to travel through the tissue. As with the transmission phase, the receiving phase may occur for a predetermined period, which may last for up to 500 microseconds. The predetermined period may depend on an expected distance from the transducers to a body cavity in the target region. The predetermined period may allow a range of 35 cm of travel, which may ensure that the device is able to capture signals so as to accurately measure bladder features for a wide range of potential users. The transducers may receive the returning echoes on the sub-apertures as described above with respect to FIG. 3A. The transducers may generate a return voltage corresponding to the returning echoes.

In some embodiments, the returning echoes may be received by the transducers using beamforming techniques. It will be understood by those skilled in the art that using beamforming techniques may better analyze the received signals and increase the accuracy of the data received and in turn, increase the accuracy of the monitoring process described herein. As the echoes are received by transducers, they may be processed by processing electronics and stored in memory of the device. Specifically, the returning echoes may be converted to digital form by the transducers and/or processing electronics, and stored in this digital form in memory. The returning echoes may be associated with a plurality of channels. The returning echoes associated with each channel may be delayed to align, or focus, the returning echoes associated the plurality of channels and summed to form a coherently summed signal. Alternatively, or additionally, the returning echoes may be transmitted to a remote device or data cloud for further processing.

At step 806, the received reflected signals may be processed to provide timing measurements associated with the body cavity. The timing measurements, or reflected echoes, may be further processed to determine bladder information. In many embodiments, the timing measurements, or the stored returning echoes, may be processed by processing electronics using signal processing algorithms to determine the bladder information. The bladder information may include the volume of fluid in the bladder, the thickness of bladder walls, the shape of the bladder, any other bladder features and/or any suitable combination thereof. As an example, the signal processing algorithms performed by processing electronics may find a distance to the front and back wall of a user's bladder along a plurality of beam directions and thus calculate the shape and volume of the bladder. In some embodiments, the processing of the returning echoes to determine bladder information may be done remotely, rather than using the processing electronics of wearable device. In this way, the processing electronics may be simplified and more powerful processors of remote devices may be utilized.

At step 808, the volume of the body cavity may be determined using the timing measurements. The volume the body cavity may be stored in memory, along with any other information measured. The determined information (e.g., bladder information) may be transmitted via wireless radio to a remote device to be stored thereon, or to be transmitted for storage on data cloud. In some embodiments, determined information may be stored temporarily and transmitted periodically to a remote device, so that space can be periodically cleared on memory for future returning echo data to be stored.

In some embodiments, the stored data may be used to visually display an indication of a user's bladder status on a remote device. For example, a mobile application may be installed on a user's remote device that may provide a user interface for the user to track bladder volume. The mobile application may provide a visual indicator of the user's bladder status, which may include the current volume in the bladder as well as an indication of a relevant threshold volume (as will be described below). In this way, the user may be able to visualize if they are coming close to a threshold amount of fluid, so that they can better manage incontinence.

In some embodiments, the mobile app may be configured to notify the user whenever the bladder status has a significant change, which may be defined as a particular percent of change in volume of the fluid in the bladder. Further, the mobile application may allow a user to look at historical data, input any additional information (such as tracking voiding events and/or cases of incontinence), and cause any of this data to be transmitted to third parties for analysis. For example, a user may want to provide historical data regarding his or her bladder status to a healthcare provider for a medical diagnosis. The mobile application may also provide a convenient way for a user to manage the device settings. For example, the user may access settings in the mobile application that determine how the alerts (described below) function, input information related to the patient that may be used in processing algorithms and threshold determination (as described below) and generally customize the operation of the device and mobile application itself.

At step 810, the volume of the body cavity is compared to one or more threshold values. For example, if the volume of the body cavity corresponds to a volume of urine in a bladder, it may be compared to a threshold volume. The threshold volume may be a volume predictive of incontinence of an individual. This may be a threshold above which an individual's ability to retain urine within the bladder is impaired. While a default threshold volume based on generally known principles may be used, in some cases, the threshold volume is unique to an individual, based on any number of factors. Accordingly, the threshold volume may be determined based upon factors such as a patient's physiological or demographic data.

Since the wearable device also collects and stores (to the device memory or the data cloud) data related to the user's bladder, this historical data, gathered using the wearable device (or other related devices), may also be used to determine the threshold volume for a given individual. For example, voiding events may be detected by the wearable device to determine a best fit volume from the bladder size associated with the voiding events. The wearable device may implement one or more machine learning algorithms, such as linear regression, to determine threshold values. Thus, the threshold volume may be dynamically changing as additional data is collected for an individual. Upon comparison of the bladder information with the threshold at step 810, it is determined whether any threshold is reached at step 812. If a threshold is not reached, the process may repeat, starting at the transmission phase described with respect to step 802.

At step 814, if it is determined that a threshold bladder volume is reached, the user may be alerted. As described above, the user may be alerted directly by the wearable device with alarm actuators, which may provide haptic alerts, visual alerts, and/or audible alerts. For example, wearable device may vibrate, may have LED indicators that flash, and/or may emit an audible tone to alert the user when a threshold bladder volume is reached. In some embodiments, wearable device may transmit an alert signal wirelessly to remote device and cause the remote device to alert the user (or a third party) as to the bladder status. For example, the wearable device may transmit a Bluetooth signal to a remote device that causes the remote device to vibrate, make a noise, flash lights, and/or display a text notification on the remote device. Once alerted, the user may be able to better manage and otherwise avoid incontinence. Upon alerting the user, the process may repeat, starting at the transmission phase described with respect to step 802. Although described herein in terms of alerting based on reaching a threshold volume, it will be understood that alerts may be customized for and by a user as described above, for example, by using the mobile application interface.

In addition to the periodic determination of bladder volume or other information described above, a wearable device may also be configured to make determinations based on other conditions. For example, in some cases, bladder volume after voiding by the user may be of particular significance. Thus, the wearable device may be able to detect a voiding event automatically (based on a decrease in fluid volume, for example) and/or receive an input from a user as to a voiding event, and the volumes before and after may be stored and marked as pre and post voiding volumes. As with the data above, this data may be stored in the device memory or remotely, and may be used to identify irregularities in voiding. For example, the processing electronics may track the determined volume after voiding and the user, medical professional, or the device itself may be able to identify irregular or dysfunctional voiding so that it can be addressed and treated appropriately.

In some embodiments, the wearable device may continuously monitor and record data associated with bladder size. The data may provide bladder information to determine how fast the bladder fills, how fast the bladder drains, and the like. Continuous activity data may be stored in the device memory or transmitted to the remoted device. In some embodiments, the device can prepare and transmit a report of bladder activity to the remote device. In other embodiments, the remote device may be configured to receive raw continuous activity data, such as volume measurements associated with the bladder, and prepare an activity report. The activity report may include information useful to the user, such as whether the bladder is full or empty. The activity report may include additional information for a physician such as fill/drain rates, volume measurements, and the like.

The wearable device may be configurable to one or more modes. For example, the wearable device can be configured to receive instructions from the remote device to begin monitoring the bladder or associated body cavity. The remote device can be configured to operate in multiple modes depending on a category of bladder dysfunction. For example, a urinary tract infection mode can configure the device to increase a number of scans during bladder activity such as urinating. The activity may be identified by an increased rate of change of the bladder volume. Another mode may be a positioning mode. In a positioning mode, the processing electronics may determine the device is positioned in a suitable location and transmit feedback to a user via alarm actuators, the remote device, and/or the personal computer.

Although devices and methods described herein illustratively refer primarily to monitoring of bladder volume in order to manage urinary incontinence, it will be understood that the devices and methods described herein are not so limited in scope. In some embodiments, the wearable device may be used as a tool to help “potty train” young children. In this case, the children may wear the device as described above, and a supervising adult may receive alerts (via a smartphone or computer as described above) when the volume of the child's bladder reaches a threshold value. The adult may then use this prediction to guide the child to void his bladder at the correct time, allowing the child to learn from these instances.

In some embodiments, the wearable device may also be used as a therapeutic device. In many medical applications, it is desirable to treat a patient with microbubbles that contain therapeutic contents. However, it is often difficult to non-invasively and precisely cause the microbubbles to release their therapeutic contents in a particular target region. Since such microbubbles may be precisely burst by ultrasonic signals as would be generated by the PMUTs described above, it will be understood that the PMUTs of the wearable device may be used to effectively release the therapeutic contents of one or more microbubbles in a desired area. Specifically, the wearable device may be positioned so that the PMUTs are aimed at a target region, and once the microbubbles reach this region, the device may be activated to burst the microbubbles and release the therapeutic contents thereof. In some embodiments, the microbubbles may be configured to act as contrast agents. The wearable device may be configured for microbubble contrast agent destruction.

### III. Volume Measurement

When using a wearable ultrasonic device to measure bladder volume, typical techniques for measuring volume are not available. Currently, imaging is done by large machines and a trained operator can identify the bladder. The machine can then determine a volume of that region from the images and input by the trained operator. A wearable ultrasonic device is provided with a small form factor, a transducer array, and processing electronics integrated with the transducer array. The integrated processing electronics and a lower resolution less than the resolution required for an image enable volume measurements to be calculated by a battery powered wearable device.

A. Device for Volume Measurement

FIG. 9 shows a simplified block diagram of a PMUT/ASIC stack according to an embodiment of the present invention. The PMUT/ASIC Stack 900 may include a PMUT stack 930 and an ASIC stack 901. The PMUT stack 930 may include a plurality of transducer channels 932. The ASIC stack may include a drive module 902, a transmit module 904, a receive module 906, a Low-Noise Amplifier/Variable Gain Amplifier (LNA/VGA) module 908, an analog-to-digital (ADC) module 910, a digital multiplexer 912, a memory 914, a signal processing module 926, a measurement module 918, a monitoring module 920, an alarm module 922, a communication module 916, a controller module 924, and a power management module 940. Some of the elements in FIG. 9 may be implement on a different chip, e.g., a master microcontroller. The elements described in FIG. 9, alone or in combination with a master microcontroller, may detect a set of peak pairs in a reflected ultrasonic signal. The elements may identify that the set of peak pairs corresponds to a set of time differences that may be used to determine the volume of a body cavity.

PMUT stack 930 shows an embodiment with four transducer channels 932, each of which may include one or more transducers. The transducer channels 932 may correspond to the elements 312 shown in FIG. 3. Each transducer channel 932 may emit ultrasonic signals toward a target region. In some embodiments, each transducer channel 932 may receive reflected signals associated with the emitted signals reflected in the target region. Each of the four transducer channels 932 may be coupled to a transmit module 904 and a receive module 906. The transmit module 904 may be configured to cause the transducers 934 to emit ultrasonic signals automatically according to a timing schedule specified in the transmit module 904 or according to commands received from the communication module 916, the controller module 924, or another device communicatively coupled to the transmit module 904. The transmit module 904 may be coupled to the drive module 902. The transmit module may include instructions that, when executed, cause the drive module to supply power to the transducer channels 932 to emit ultrasonic signals.

In some embodiments, the transmit module 904 may be configured to include a steering module. The steering module may shift the transmitted pulses to steer the ultrasonic beam emitted from the transducer channels 932. The transmit module 904 may steer the emitted pulses in the elevation direction and the azimuth direction.

The combined signal generated from transducer channels 932 can form beams (lines), e.g., due to the constructive and destructive effects of the difference wave patterns of the individual transducers. The lines can be defined based on the positions of transducer channels 932.

After emitting an ultrasonic signal, the receive module 906 may begin receiving an analog signal in response to the reflected ultrasonic signals received at the one or more transducer channels 932. In some embodiments, the transducer channels 932 may correspond to a plurality of channels. Each transducer channel 932 can receive echoes from different points of the body cavity, e.g., corresponding to the different beams (lines) that are emitted. Each echo can correspond to a peak in the reflected signal, and the echoes from a given point of the body cavity will reach the different channels at different times. Accordingly, the receive module 906 may include a steering module to introduce a delay in the received analog signal.

The receive module 906 may transmit the analog signal to the an amplifier module such as LNA/VGA Module 908. The LNA/VGA module 908 may amplify the analog signal and transmit an amplified analog signal to the ADC module 910. The receive module 906 and LNA/VGA module 908 may be operable to boost the signal to noise ration of the received analog signal. The ADC module 910 uniformly samples the amplified analog signal and transmits the digital signal to the memory module 914. In other embodiments, the ADC module may transmit the digital signal to the digital multiplexer 912. The digital multiplexer 912 may transmit the digital signal to one or more of the signal processing module 926, the communication module 916, and the memory 914. In some embodiments, memory module 914 is a FIFO memory. Data signals for a plurality of channels stored in the memory module 914 may correspond to a selected focal point. The memory module 914 may include a 256 KB internal buffer.

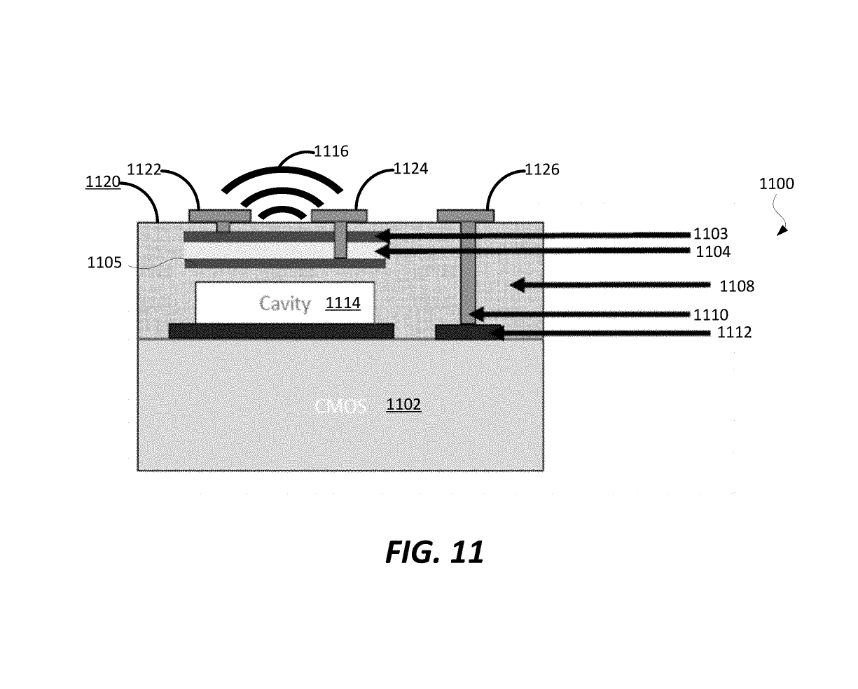

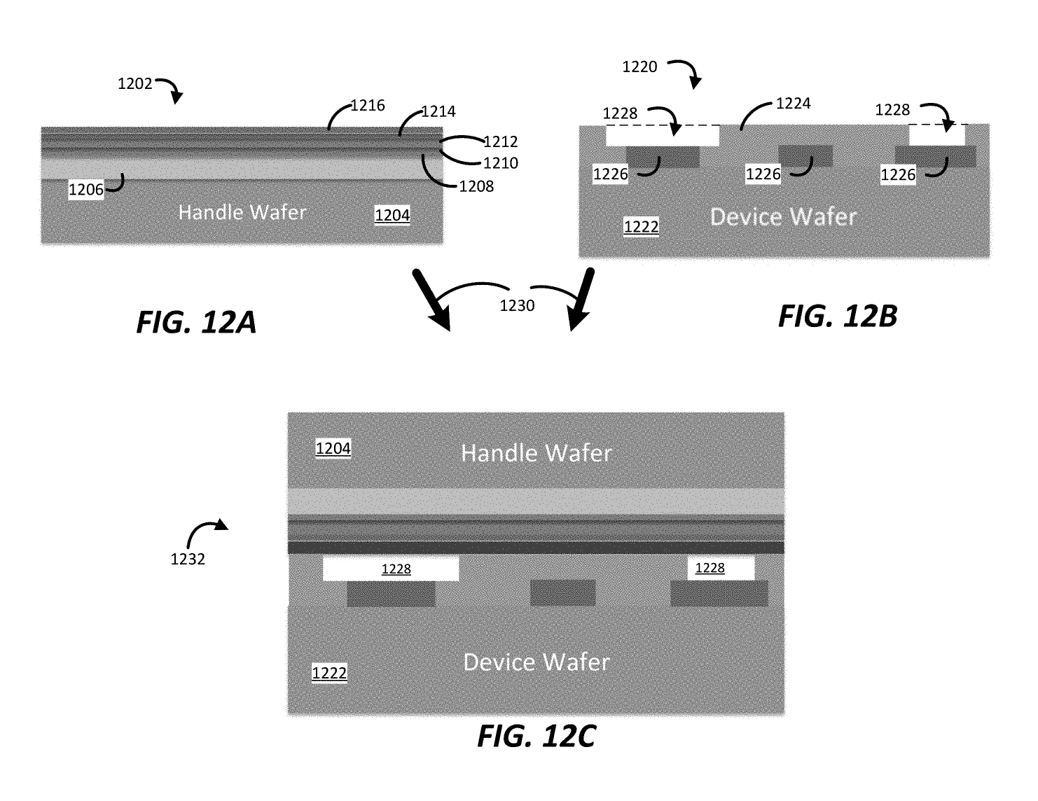

The signal processing module 926 may receive digital signals for each channel from at least one of memory module 914, digital multiplexer 912, and communication module 916. The signal processing module 926 may receive or include data or circuitry to implement a delay profile (which may be predetermined) for the incoming digital signals, where the delay profile is used align the data signals for a given line. For greater accuracy, multiple channels can be used to measure a same point on a body cavity. To combine the measurements, delays can be used to align the signals to provide a single measurement. The alignment is needed since the channels are at different positions, and thus the echo from a point will reach the different channels at different times, hence the delays. Further details are described with respect to FIGS. 10A-10C.

The delayed signals or measured values from the delayed signals (e.g., timing values) may be summed to form an average measurement, e.g., as digital data. In some embodiments, the signal processing module 926 may detect an envelope of a waveform associated with the digital data. The envelope may be logarithmically compressed and processed to improve the signal to noise ratio of the digital data. The digital data may include peaks with a reception time that may be used to determine a time of flight of the ultrasonic signal to various points on the body cavity. Envelope detection may simplify peak detection. The signal processing module 926 may transmit the digital data to the measurement module 918 to determine properties related to a body cavity in the target region. The digital data can include an averaged time measurement for echoes from each of a plurality of points along emitted lines.

The measurement module 918 may include instructions and/or circuitry to determine one or more of the presence of a body cavity in the target region, the dimensions of the body cavity, the volume of the body cavity and the like. In some embodiments, the measurement module may be configured to determine the rate of change of various physical properties related to the body cavity. In some embodiments, the cavity is a user's bladder.

To perform such measurements, measurement module 918 can identify a pair of peaks (e.g., by analyzing times associated with the peaks) that correspond to a same line. The pairs of peaks can be determined based on an expected timing relationship. For example, the peak (pulse) times can form two sets, with the first set being received first and associated with a front wall. A second set of times can correspond to a back wall and be received later. Further details are described with respect to FIGS. 10A-10C.

The measurement module may transmit body cavity measurements to a monitoring module 920. The monitoring module 920 may monitor body cavity properties to determine if a particular property crosses a threshold value. Examples of threshold values can include a minimum volume, a warning volume, a maximum volume, a maximum rate of change value, and the like. The monitoring module 920 may determine one of the threshold values has been reached or passed and transmit an alarm condition to alarm module 922. The alarm module 922 may activate an alarm system coupled to the processing board of the wearable device. For example, the alarm module 922 may activate a speaker, an LED, or a haptic feedback module to alert the user of an alarm condition. In some embodiments, the alarm module 922 may be coupled to the communication module 916 and be configured to cause an alarm condition on a connected mobile device such as a smart watch, mobile phone, tablet, laptop computer, and the like.

In some embodiments, the monitoring module 920 may detect conditions bladder dysfunction. For example, if the minimum volume threshold is not reach, the alarm module 922 may notify the user of an incomplete evacuation of the bladder and the user is at an increased risk of a urinary tract infection. In another example, if the warning volume is reached, the alarm module 922 may transmit an alert to notify a user suffering from urinary incontinence of a pending enuresis. In yet another example, if the maximum rate of change value is exceeded, the alarm module 922 may notify the user may be alerted of ongoing enuresis. In some embodiments, the thickness of a wall of the bladder may be determined.

The ASIC 901 may further include a power management module 940. The power management module 940 may interface with the battery on the processing electronics board. The power management module 940 may include various output voltages depending on the requirements of the modules coupled to one or more outputs. In some embodiments the power management module operates at 24 V. The power management module 940 may provide a 24 V signal to the drive module 902 in order to drive the transducer channels 932 in the PMUT stack 930. The drive module 902 may drive the transducers at a voltage less than approximately 4 V.

In some embodiments, the ASIC 901 may include a controller module 924. The controller module 924 may include instructions to execute one or more of the modules implemented in the ASIC 901. In some embodiments, one or more of the modules, such as the signal processing module 926, the measurement module 918, the monitoring module 920, the alarm module 922, and the like may be implemented in the master microcontroller 510 described in FIG. 5A. The communication module 916 may be configured to transmit data using a general purpose serial interface, such as a 7-wire communications interface. One of ordinary skill in the art would recognize many variations, modifications, and alternatives.

B. Beamshaping Using Transducer Array

In some embodiments, a method is provided to measure a volume using timing measurements corresponding to different lines through the bladder. Specifically, a transducer can emit an ultrasonic signal, which expands as a wave pattern in the body. The reflections (echoes) from the front wall of the bladder may be measured first, and reflections from the back wall second. Each line may provide two measurements: one for the front side of the bladder and one for the backside of the bladder. The lines can be generated by an array of transducers.

FIG. 10A shows a simplified diagram of processing a reflected ultrasound wave 1036 on a plurality of channels according to an embodiment of the present invention. In some embodiments, an array of transducer channels 1060 receive raw data 1062 associated with a reflected ultrasound wave 1036. The raw data is processed using the signal processing module 1064 to determine a plurality of scan lines 1066. The signal processing module uses one or more delay profiles to determine the plurality of scan lines 1066 from the raw data 1062. The scan lines 1066 may be processed to discard any unsatisfactory readings. The scan lines may be compared to a threshold value prior to being considered for the volume calculation.

An emitted pulse can be generated using the array of channels 1060. Each channel 1070 may comprise 49 transducers. The array of channels 1060 may be excited at the same time to emit a pulse directed toward a target area. The aperture size may correspond to the size of the array of channels 1060. In some embodiments, a device may include 16 channels on a tile and 8 tiles in the device resulting in 128 channels. For example, when a plurality of transducers are used, the resulting wave patterns of the array combine to provide linear signals. The wave patterns essentially cancel out except along the lines (e.g., 231 lines). The exact position and number of transducers affects the number of lines and their position. Thus, each of the detected pulses 1072 correspond to one of the lines. The received signal 1074 can be analyzed using a signal processing module 1064 to identify the times of the pulses 1072, and match pulses as corresponding to a same line. This matching can be done based on an expected range of reception times for a given line. The signal processing module 1064 can include a delay set for each channel, e.g., where these delays correspond to different time windows in which the peak for a particular point (front or back wall) of a line is expected to be found. The delay profile may include delays (time windows) associated with each line. In some embodiments, the delay (time window) profile may include 231 different delays (time windows) per channel. These delays for different peaks (pulses) that correspond to different points along beam lines through the body cavity should not be confused with the delays associated with echoes from a single point to multiple channels, where such other delays are used for aligning signals to obtain an averaged timing value for that point. Thus there are delays between lines for a given channel (corresponding to time windows), and delays between channels for a given point on a line.

The reflected signal can include a first set of separate pulses 1078 corresponding to different point on the first wall 1030. Each of the first set of separate pulses 1078 corresponds to a different delay time relative to emission, where a longer time delay corresponds to a front wall point farther from the transducer and a shorter time delay corresponds to a front wall point closer to the transducer.

The delay between emission and receiving the pulses for a given point (pixel) of a bladder wall can be determined by the device, e.g., by analyzing a channel signal over a time window corresponding to an expected time. The time window can be updated, if a peak is not identified. For example, the distance to the closest front wall point can be a fixed proportion relative to the distance from the transducer to that closest point.

Further, the time measurement for a given point can be averaged over multiple channels. Each channel will receive the echo from a point at a different time. But, an expected delay (i.e., between channel, not between lines) can be determined based on the distance between channels. After this compensation (aligning) a single time measurement 1076 can be obtained as an average of the time measurements for channels having an appreciable peak in a time window corresponding to that channel. The single time measurement can be determined by coherently summing the aligned raw data. Summation of the aligned raw data can focus the returning echoes and improve the signal to noise ratio by eliminating the noise that may appear on an individual line. Using the raw data 1062 or the single time measurement 1076, the wearable device can detect an envelope 1080 associated with the peaks. The envelope 1080 may be detected using various signal processing techniques such as an analytic signal detector, and the like. Envelope 1080 may simplify peak detection to determine a peak pair 1071. The peak pair 1071 may comprise a peak associated with a front echo that corresponds to pulses 1072, and a second peak associated with a back echo 1073. The received echoes may be further processed to determine a first side 1084 and a second side 1086 of the bladder wall. The wall thickness 1082 may be determined using the first side 1084 and the second side 1086. The wall thickness may be stored in memory or transmitted to a remote device.

C. Emission Lines Per Channel

FIGS. 10B and 10C illustrate return echoes associated with a body cavity disposed in a target area, or range of coverage, for a channel. FIG. 10B illustrates a return echo associated with a first body cavity disposed in the range of coverage of a channel according to an embodiment of the present invention. FIG. 10B shows a channel 1000 at a contact point 1002 with a user's skin 1004. The user's skin 1004 may be at the abdominal surface near a user's bladder 1032. The channel 1000 may transmit a single pulse ultrasound wave. The ultrasound wave may be characterized by both a pulse duration and a pulse frequency. A reflected ultrasound wave 1036 may be received at the channel. The channel 1000 can monitor the reflection coming off of a first wall 1030 of the bladder 1032 and a second wall 1034 of the bladder 1032. The reflected ultrasound wave 1036 may include a first peak 1038 and a second peak 1040 corresponding to the first wall 1030 and the second wall 1034 respectively. The peaks of the reflected ultrasound wave 1036 may correspond to a time of flight of an ultrasound pulse emitted from the channel 1000 to the first wall 1030 and the second wall 1034. A time of flight between the first wall and the second wall 1042 may be used to determine the volume of the bladder. The time of flight between the first wall and the second wall 1042 can correspond to the first peak 1038 and the second peak 1040 and be used to determine a distance between the first wall 1030 and to the second wall 1034 of the bladder 1032. In other embodiments, a first distance from the channel 1000 to the first wall 1030 and a second distance from the channel 1000 to the second wall 1034 may be determined to calculate the volume of the bladder.

In some embodiments, the reflected ultrasound wave 1036 may be received by a plurality of channels. The reflected ultrasound wave 1036 may be sampled at each channel by an analog-to-digital converter and stored in a first-in-first-out (FIFO) memory. The measurement and processing circuitry may select data from the FIFO memory that corresponds to a focal point. In some embodiments data from a plurality of channels may be selected and summed for reflected ultrasound wave 1036 beamforming. Beamforming can be used to form a plurality of lines 1022. The reflected ultrasound wave 1036 beamforming may be performed by a signal processing module. The signal processing module may receive digitized echoes for each channel. The digitized echoes may be delayed based on a pre-determined delay profile and coherently summed to form digital scanline data, or, put simply, lines.

While a volume for the body cavity may be determined using a single line, to enhance the accuracy the plurality of lines 1022 may be used. The plurality of lines 1022 can be formed using digitized echoes for each channel and adjusting the delay profile to form a different line. The echo arrives at each channel at different times depending on the location of a target point in the target area and the channel position on the transducer. The center channel receives the echo from the target point at a reference time different from the off-center channels. The pre-determined delay profile may include channel delays corresponding to a time delay for each channel relative to the center channel. For example, the delay for a specific target point in the target area for the center channel may be delay_{point n}. The delay for each off-center channel may be the delay for the specific target point plus an additional delay associated with the off-center channel, delay_{point n}+delay_{off-center channel n}. By applying the proper time delay to each channel, the received echoes may be aligned before being coherently summed. Aligning the received echoes may provide a focused reflected signal. In some embodiments, a resolution of the number of lines used to determine the volume of the bladder 1032 may be less than the resolution required for a visual image. The lower resolution may be associated with a lower device voltage. The lower device voltage may improve the battery life of the wearable device. In some embodiments, a plurality of lines can be spaced according to a focal distance.

In some embodiments, to resolve the size of a body cavity, a particular resolution is necessary. The particular resolution may be lower than a resolution required to image the bladder 1032. Ultrasound resolution depends on the size of the active aperture of the transducer or transducer array, the frequency and bandwidth of the transducer, and the selected transmit focal distance. the range of coverage can be determined by the aperture size. A synthetic aperture can be produced using a multi-channel ultrasound transducer with digital beamforming. The aperture may be larger than an object of interest in the range of coverage. The range of coverage can be characterized by a focal distance 1050, elevation line spacing 1052, and azimuth line spacing 1054. In some embodiments, the bladder 1032 is disposed in the range of coverage. FIG. 10B illustrates a voided bladder 1032 in the range of coverage. The voided bladder can be approximately 1.5 cm tall by 4 cm wide by 5 cm deep. The distance from the user's skin to the voided bladder may be 5 cm to the first wall 1030 and 10 cm to the second wall 1034.

In some embodiments, the focal distance 1050 may be 10 cm. In the z-direction 1090, the elevation line spacing 1052 may be approximately 0.5 cm at the focal distance 1050. In some embodiments, a target area of 10 cm in the z-direction 1090 may be used. With an elevation line spacing of approximately 0.5 cm, 21 lines may be used to cover the target area. In the y-direction 1092, the azimuth line spacing 1054 may be 1 cm at the focal distance. In some embodiments, a target area of 10 cm in the y-direction may be used. With an azimuth line spacing in the y-direction of approximately 1 cm, 11 lines may be used to cover the target area of 10 cm in the y-direction. In with the elevation and azimuth spacing described, 231 total lines may be used. The max steer angle for a system with 231 lines may be 26.6°. Beamforming and signal processing may generate an approximately 10 nanosecond resolution. In some embodiments, lateral resolution requirements at a focal distance of 10 cm may include λ/D≤0.5 in elevation and 0.1 in azimuth, where D is the focal distance in cm and λ is the wavelength in MHz. In some embodiments, using 231 lines, the volume of a body cavity may be determined within a standard deviation of 5 cc.

FIG. 10C illustrates a return echo associated with a second body cavity disposed in the range of coverage of a channel according to an embodiment of the present invention. In FIG. 10C, the second body cavity is a full bladder 1033. An average full bladder 1033 may be 7 cm tall by 8 cm wide by 11 cm deep. The distance from user's skin 1004 may be 2.5 cm to the first wall 1030 and 13.5 cm to the second wall.

As shown in FIG. 10C, the position of the front wall and the second wall can change relative to the position of the channel 1000. To address changes in target position, a calibration phase may be implemented by the a controller module and a signal processing module. The focal distance 1050 may be adjusted to improve the detection of the peaks at a particular distance from the channel 1000. The focal distance may correspond to a time window in the received digital data. For example, the calibration phase may begin with a focal distance at a first distance. The reflected ultrasound wave associated with the first distance may be processed by the signal processing module and determine if a suitable echo is present. If a suitable echo is not detected, the controller module may shift the focal distance to a second distance. A ultrasound wave is transmitted and received. The second reflected ultrasound wave associated with the second distance may be processed by the signal processing module. If a suitable echo is present, the signal processing module may begin the volume measurement processing and calculation using the second reflected ultrasound wave. The signal processing module and the controller module can be configured to continue shifting the focal distance until a suitable echo is received. The wearable device can customize the calibration phase for a particular user based on the data collected during continuous measurement. The calibration phase can update the delay profile with delays associated with the lines for each channel.

In some embodiments, the calibration phase can detect lines not directed at the target. Line 1056 in FIG. 10C does not scatter off the full bladder 1033 in any body cavity state, e.g., when the bladder is full or empty. The calibration phase may determine, based on an estimated center of the cavity, missed lines and discard the data from time and volume calculations. In some embodiments a channel may be positioned such that no lines associated with the channel intersect the target. In these cases, the wearable device may discard all data associated with the channel. In addition to missing the target, the calibration phase may determine data from a channel or line is erroneous and discard the associated data.

### IV. Composition of Transducer/Processing Device

The wearable electronic device may include transducer circuitry coupled to processing circuitry in a combined device. The transducer circuitry may include one or more piezoelectric micromachined ultrasonic transducers (PMUTs). The transducer circuitry may be provided on a first substrate and the processing circuitry may be formed on a device wafer. A plurality of cavities may be formed on the device wafer to isolate the processing circuitry from ultrasonic signals generated by the transducer circuitry. The device wafer may be formed using standard CMOS processes. The stacked CMOS device may be formed by bonding the first substrate to the device wafer and forming contacts thereon. The stacked CMOS device may operate at lower voltages.

A. Transducer Device Components

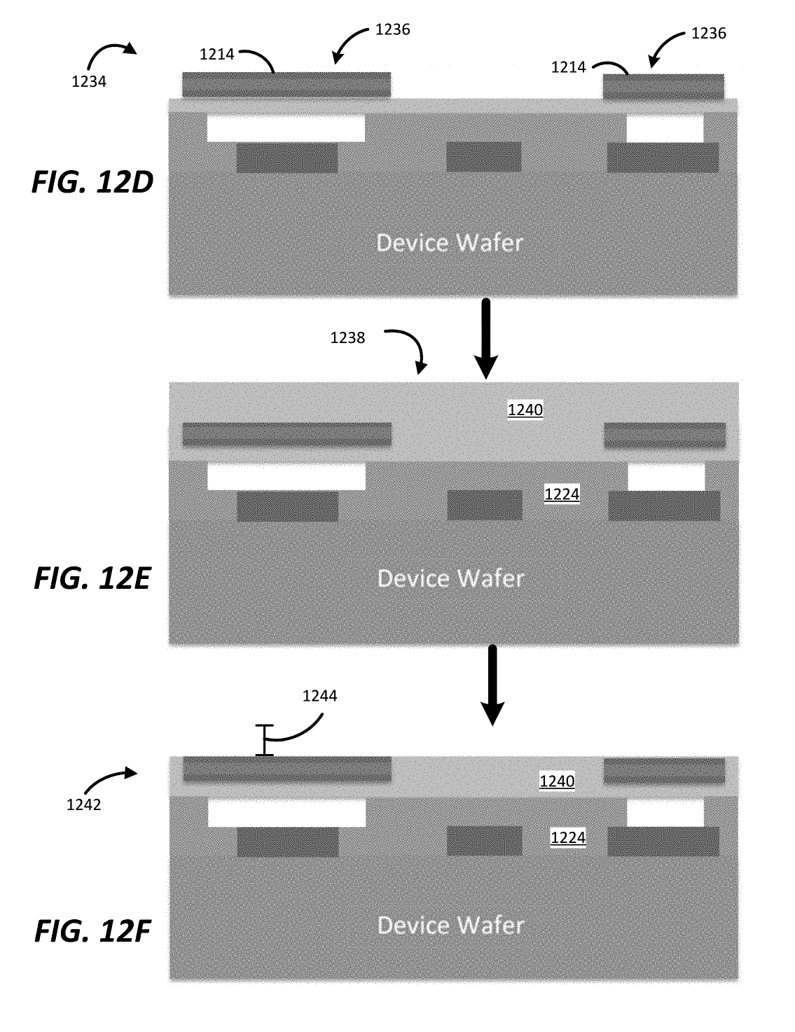

FIG. 11 shows a cross-sectional view of a single piezoelectric micromachined ultrasonic transducer 1100 bonded to a CMOS wafer 1102 used in a wearable device as described above, in accordance with many embodiments. As described above, and as shown in FIG. 11, each piezoelectric micromachined ultrasonic transducers (PMUT) 1100 may be formed on a MEMS wafer that is bonded to a CMOS wafer 1102. In this way, PMUT 1100 may be coupled to the requisite processing electronics of the CMOS wafer 1102. It will be understood that each PMUT may have an active piezoelectric layer 1104 along with a first electrode 1103 and a second electrode 1105. The first electrode 1103 and the second electrode can be electrically coupled to the piezoelectric layer 1104.

In some embodiments, the PMUT 1100 may include a first contact 1122 electrically coupled to the first electrode 1103, a second contact 1124 electrically coupled to the second electrode 1105, and a third electrode electrically coupled to the CMOS wafer 1102. In some embodiments, a bias may be introduced between the first electrode 1103 and the second electrode 1105 to cause stress in the piezoelectric layer 1104. Alternating the bias at a predetermined frequency may cause the piezoelectric layer 1104 to generate sound waves. One or more vias 1110 may be formed to in the PMUT 1100. Each of the contacts may be wire bonded outside PMUT 1100 to an electronics board. In some embodiments, PMUT 1100 may include a passivation layer formed on a surface 1120 and the contacts. Referring to FIG. 1, the top surface 210 of an adhesive coupling interface may be coupled to the surface 1120 of the PMUT 1100.

In some embodiments, the passive elastic layer 1108 may comprise SiO₂ or any other suitable passive layer. The active piezoelectric layer 1104 may be approximately 1 μm thick Aluminum Nitride, and the passive elastic layer may be approximately 1 μm thick single-crystal Silicon. In some embodiments, the active piezoelectric layer 1104 may be Scandium-doped Aluminum Nitride. Alternatively, the active piezoelectric layer 1104 may be another suitable piezoelectric ceramic such as PZT. Both the top and bottom electrodes 1106 may comprise Molybdenum. In order to bond the PMUT 1100 to the top metal 1112 of CMOS wafer 1102, fusion bonding via thru-silicon-via (TSV) as shown at 1110 may be used. This methodology results in significant parasitic reduction which in turn results in generation of increased acoustic intensity in operation.

In some embodiments, cavity 1114 may be formed with a vacuum or near vacuum to isolate the transducer from the processing electronics in the CMOS wafer 1102. The sound generated by PMUT will not travel through the cavity 1114 minimizing reflection and interference that may be caused by material interfaces with the CMOS wafer 1102. The cavity 1114 may cause ultrasound 1116 to travel away from the PMUT 1100. Ultrasound 1116 may travel through the adhesive coupling interface and into the body of a user. The body, and the bladder, may reflect ultrasound 1116 causing a return echo to reflect back to the PMUT 1100. The return echo travels through the adhesive coupling interface and is received by the PMUT 1100.

After transmitting ultrasound 1116, the bias between the first electrode 1103 and the second electrode 1105 can be removed. The return echo may cause stress in the piezoelectric layer 1104. The stress causes an electric charge to accumulate in the piezoelectric layer 1104. In some embodiments, the first electrode 1103 and the second electrode may measure the electric charge that accumulates in the piezoelectric layer.

In some embodiments, the CMOS wafer 1102 may be an application specific integrated circuit (ASIC) that includes one or more devices necessary to drive the transducer. The drive voltage for an array of PMUTs may be less than 4 volts. The ASIC can be manufactured to meet size requirements associated with the size of an associated PMUT. In some embodiments, the ASIC may include one or more modules to receive measured signals. The ASIC may be configured to further process the signal. For example, the ASIC may include one or more analog to digital converters to convert the returning echoes to a digital signal. In some embodiments, the ASIC may transmit the digital signal to at least one or more of a memory, a processor, and a remote device. In other embodiments, the ASIC may include one or more signal processing modules.

B. Manufacturing Process to Create Transducer, Layers and Materials.

FIGS. 12A-12H illustrate a device stack corresponding to the process steps used to fabricate a piezoelectric micromachined ultrasonic transducers (PMUTs) bonded to an application specific integrated circuit (ASIC) according to an embodiment of the present invention. Bonding the plurality of PMUTs to an ASIC reduces parasitics and the bonded PMUTs can be driven at a low voltage, for example, less than 4 volts. The ASIC may include circuitry and/or instructions to execute one or more of the modules shown in FIG. 9. To form an integrated PMUT/ASIC stack, several steps may be performed. FIG. 12A shows a transducer device 1202. The transducer device 1202 may comprise a first wafer 1204, an oxide layer 1206, a seed layer 1208, a first electrode layer 1210, a piezoelectric layer 1212, a second electrode layer 1214, and a bonding oxide layer 1216. The first wafer 1204 may be referred to as a handle wafer, a first substrate, and the like. In some embodiments, the first wafer 1204 is a silicon wafer that may be removed during one or more processing steps. An oxide layer 1206 may be formed on the first wafer 1204. In some embodiments, the oxide layer 1206 may be a 500 Å thermal oxide layer. In some embodiments, the first wafer 1204 may be a first substrate and provided with an oxide layer 1206 formed on a handle wafer. Next, a seed layer 1208 is formed on oxide layer 1206. The seed layer 1208 may be aluminum nitride (AlN). The seed layer 1208 may be used to influence the crystal structure of layers deposited thereon. In some embodiments, the first substrate may include the seed layer 1208.

The transducer device 1202 may include a plurality of PMUT device layers. The first PMUT device layer may include a first electrode layer 1210 coupled to the seed layer 1208. In some embodiments, the thickness of the first electrode layer 1210 deposited on the seed layer 1208 may be approximately 200 Å. The first electrode layer 1210 may be molybdenum. The crystal structure of the first electrode layer 1210 may be determined by the grain boundary with the seed layer 1208. The grain boundary may depend on the crystal structure of the seed layer 1208.

A piezoelectric layer 1212 may be coupled to the first electrode layer 1210. The piezoelectric layer 1212 may include one or more of AlN, Scandium-doped AlN, Lead Zirconate Titanate (PZT), and the like. In some embodiments, the thickness of the piezoelectric layer 1212 deposited on the first electrode layer 1210 may be approximately 1 μm. In other embodiments, the piezoelectric layer 1212 can comprise a plurality of layers such as a first platinum layer, a PZT seed layer, a PZT layer, and a second platinum layer. The thickness of each of the first platinum layer and the second platinum layer may be approximately 200 Å, the thickness of the PZT seed layer may be approximately 30 Å, and the thickness of the PZT layer may be approximately 2 μm. The crystal structure of the piezoelectric layer 1212 may be determined by the crystal structure of the seed layer 1208. In some embodiments, the first electrode layer 1210 crystal structure may approximately align with the crystal structure of the seed layer 1208. The grain boundary between the first electrode layer 1210 and the piezoelectric layer 1212 may determine the crystal structure of the piezoelectric layer 1212.

The PMUT device layers may further include a second electrode layer 1214 coupled to the piezoelectric layer 1212. In some embodiments, the thickness of the second electrode layer 1214 deposited on the piezoelectric layer 1212 may be approximately 200 Å. The second electrode layer 1214 may be molybdenum. The crystal structure of the second electrode layer 1214 may be determined by the grain boundary with the piezoelectric layer 1212. The grain boundary may depend on the crystal structure of the piezoelectric layer 1212. After forming the PMUT device layers, a bonding oxide layer 1216 may be formed on the second electrode layer 1214. The bonding oxide layer can be a high density plasma (HDP) oxide. In some embodiments, the thickness of the bonding oxide layer deposited on the second electrode layer 1214 can be approximately 500 Å.

FIG. 12B shows a second portion of the integrated PMUT/ASIC stack may include a processing device comprising a device wafer 1220. The device wafer 1220 may include a “system-on-a-chip” ASIC 1222. The ASIC 1222 may be built using standard semiconductor processing steps, such as 0.18 μm CMOS. The ASIC 1222 may include general and signal processing circuitry, measurement circuitry, alert circuitry, memory, and the like. The ASIC may include one or more contacts 1226 coupled to the electrical circuits of the ASIC 1222. The contacts 1226 may be a conducting material, for example, aluminum, copper, gold, and the like. In some embodiments the contacts 1226 may be bond pads associated with the circuitry of the ASIC 1222. A device oxide layer 1224 can be formed on the ASIC 1222 and the contacts 1226. The device oxide layer 1224 may form a passivation layer for the ASIC 1222 and contacts 1226. The thickness of the device oxide layer 1224 deposited on the ASIC 1222 and contacts may be 5000 Å. The device oxide layer 1224 may be an oxide layer. In some embodiments, the passivation layer may be deposited using Plasma Enhanced Chemical Vapor Deposition (PECVD). After depositing the device oxide layer 1224, device wafer 1220 may be patterned using well known semiconductor techniques. The device oxide layer 1224 may be patterned to produce vias to access the contacts 1226 or form one or more cavities 1228 where the transducers are to be formed.

FIG. 12C shows the transducer device 1202 bonded to the device wafer 1220. To form the integrated PMUT/ASIC stack 1232 the transducer device 1202 may be bonded to the device wafer 1220. The bonding oxide layer 1216 of the transducer device 1202 may be fusion bonded with the device oxide layer 1224 of the device oxide layer 1224 of the device wafer 1220. The surfaces may be prepared for fusion bonding by dry cleaning or wet cleaning to remove any impurities. In some embodiments, the transducer device 1202 and the device wafer 1220 may be fusion bonded by annealing the integrated PMUT/ASIC stack 1232 at approximately 300° C. for 3 hours. The fusion bonding may be performed at low pressure or near vacuum to form a vacuum in the one or more cavities 1228. The vacuum may facilitate directing sound waves generated by the PMUT away from the ASIC 1222.

After forming the PMUT/ASIC stack 1232, the first wafer 1204, the oxide layer 1206, and the seed layer 1208 may be removed from the stack 1234 as shown in FIG. 12D. The layers may be removed by back grinding until the PMUT device layers are exposed. Back grinding may be used to expose the second electrode layer 1214. The remaining PMUT device layers may be patterned to form one or more transducer areas 1236. A patterned PMUT/ASIC stack 1238 may have an oxide layer 1240 formed thereon as illustrated in FIG. 12E. The oxide layer 1240 may electrically isolate the one or more transducers 1236. In some embodiments, the oxide layer 1240 may be deposited using PECVD. The oxide layer 1240 may be approximately 3000 Å. FIG. 12F shows the PMUT/ASIC stack after a portion of the oxide layer 1240 is removed. After depositing oxide layer 1240, the second electrode layer 1214 may be exposed on PMUT/ASIC stack 1242 by removing a portion 1248 of oxide layer 1240. The oxide layer 1240 may be removed using chemical mechanical polishing.

FIG. 12G illustrates a PMUT/ASIC stack 1244 with one or more vias. After exposing the second electrode layer 1214, one or more vias may be formed in the PMUT/ASIC stack 1244. A first set of vias 1246 may be patterned to provide contact with the first electrode layer 1210. The first set of vias 1246 may be formed by etching through the second electrode layer and the piezoelectric layer to expose the first electrode layer. A second set of vias 1248 may be patterned to provide contact with the one or more contacts 1226 coupled to the electric circuitry of the ASIC 1222. The second set of vias 1248 may be formed by etching through the oxide layers 1240, 1224.

FIG. 12H illustrates a finished PMUT/ASIC stack. The stack can include device contacts and a passivation layer. To finish the device, conductor channels may be formed by depositing conductive material in the one or more vias formed in PMUT/ASIC stack 1244. The conductive material may be one or more of Ti/TiN, Al, Au, Ag, and the like. In some embodiments, 200 Å of Ti/TiN may be deposited, followed by the deposition of 9000 Å of Al. The deposited conductive material may be patterned to form the one or more device contacts. The device contacts may be patterned to form one or more bond pads at surface 1258. A first electrode device contact 1250 and a second electrode device contact 1252 may be formed for each transducer area 1236 by the conductor channels. One or more ASIC device contacts 1254 may also be formed by depositing and patterning the conductive material. After patterning the conductive material, a passivation layer 1256 may be formed on surface 1258 of the oxide layer 1240 to electrically isolate the device contacts. In some embodiments, the passivation layer 1256 may be SiO₂. In some embodiments, the thickness of the passivation layer 1256 may be approximately The passivation layer 1256 may be etched to expose the one or more device contacts.

The dimensions of the cavity may be determined to produce echoes at a particular frequency. In some embodiments, the PMUT may transmit at a frequency of for example, 2 MHz, 3.5 MHz, 5 MHz, or higher to produce data suitable for image formation. In some embodiments, the PMUT can transmit at frequencies less than 1 MHz to produce data suitable to determine the depth of a bladder wall. The PMUT may be configured to transmit at a range of ultrasound frequencies from 20 KHz to 20 MHz.

The methods and techniques described herein may be used to form a variety of PMUT arrays compatible with CMOS semiconductor processes. In some embodiments, PMUT materials and dimensions can be compliant with Semiconductor Equipment and Materials International (SEMI) standard specifications. Because PMUTs can be compliant with SEMI specifications, the transducer arrays can be used with existing CMOS semiconductor fabrication tools and methods. For example, in some embodiments, photolithography may be used to form one or more PMUTs. In contrast, current piezoelectric ultrasound transducer arrays are formed using a die saw that cannot match the precision of photolithography. As a result, PMUTs can be smaller, operate at lower voltages, and have lower parasitics.

### V. Example Computer System

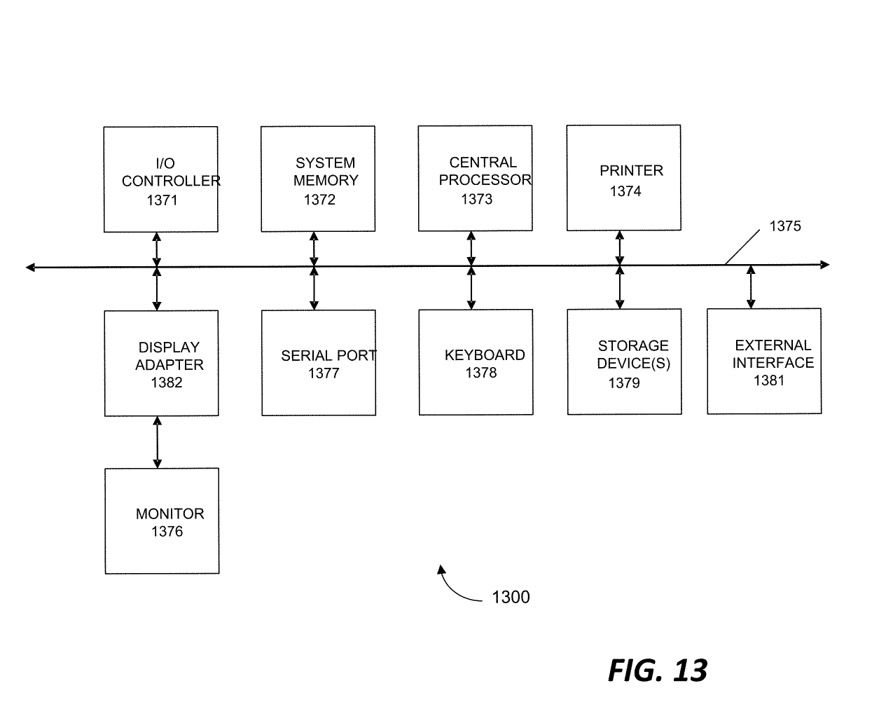

Any of the computer systems mentioned herein may utilize any suitable number of subsystems. Examples of such subsystems are shown in FIG. 13 in computer apparatus 1300. In some embodiments, a computer system may include a single computer apparatus, where the subsystems can be the components of the computer apparatus. In other embodiments, a computer system can include multiple computer apparatuses, each being a subsystem, with internal components. Aspects of the computer system may be used as part of the wearable device, the processing electronics, and the application specific integrated circuit described above.

The subsystems shown in FIG. 13 are interconnected via a system bus 1375. Additional subsystems such as a printer 1374, keyboard 1378, storage device(s) 1379, monitor 1376, which is coupled to display adapter 1382, and others are shown. Peripherals and input/output (I/O) devices, which couple to I/O controller 1371, can be connected to the computer system by any number of means known in the art such as input/output (I/O) port 1377 (e.g., USB, FireWire®). For example, I/O port 1377 or external interface 81 (e.g. Ethernet, WiFi, etc.) can be used to connect computer system 1300 to a wide area network such as the Internet, a mouse input device, or a scanner. The interconnection via system bus 1375 allows the central processor 1373 to communicate with each subsystem and to control the execution of a plurality of instructions from system memory 1372 or the storage device(s) 1379 (e.g., a fixed disk, such as a hard drive, or optical disk), as well as the exchange of information between subsystems. The system memory 1372 and/or the storage device(s) 1379 may embody a computer readable medium. Another subsystem is a data collection device 1385, such as a camera, microphone, accelerometer, and the like. Any of the data mentioned herein can be output from one component to another component and can be output to the user.

A computer system can include a plurality of the same components or subsystems, e.g., connected together by external interface 1381, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer program product (e.g. a hard drive, a CD, or an entire computer system), and may be present on or within different computer program products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The above description of example embodiments of the invention has been presented for the purposes of illustration and description. It is not intended to be exhaustive or to limit the invention to the precise form described, and many modifications and variations are possible in light of the teaching above.

All references, including publications, patent applications, and patents, cited herein are hereby incorporated by reference to the same extent as if each reference were individually and specifically indicated to be incorporated by reference and were set forth in its entirety herein.

1. A wearable ultrasonic device, the device comprising:
a coupling pad interface for affixing the device to a surface of a user's body and providing acoustic coupling;
at least one ultrasonic transducer coupled to the coupling interface and configured to:
emit ultrasonic signals directed towards a target region, and
receive returning ultrasonic signals associated with the emitted ultrasonic signals;
processing electronics coupled to the at least one ultrasonic transducer; and
a battery coupled to and configured to power the ultrasonic transducer and processing electronics.

2. The wearable ultrasonic device of claim 1, wherein the processing electronics are further configured for use in a non-invasive determination of a volume of a fluid in a body cavity associated with the returning ultrasonic signals.

3-4. (canceled)

5. The wearable ultrasonic device of claim 1, further comprising a wireless radio coupled to the processing electronics and configured to transmit stored information associated with the returning ultrasonic signals to a remote computing device, the remote computing device being configured to determine a volume of a fluid in a body cavity based on the stored information.

6. The wearable ultrasonic device of claim 1, wherein the processing electronics are configured to store information associated with the returning ultrasonic signals and determine a volume of a fluid in a body cavity based on the stored information.

7. The wearable ultrasonic device of claim 2, wherein the processing electronics are configured to compare the determined volume of the fluid with a threshold volume and output an alert when the determined volume exceeds the threshold volume.

8-11. (canceled)

12. The wearable ultrasonic device of claim 5, wherein the processing electronics are configured to receive control signals, user-specific data, or both from a remote device via the wireless radio.

13-21. (canceled)

22. The wearable ultrasonic device of claim 1, wherein the at least one ultrasonic transducer comprises an array of piezoelectric micromachined ultrasonic transducers (PMUTs).

23-30. (canceled)

31. The wearable ultrasonic device of claim 1, wherein the at least one ultrasonic transducer is configured to emit and receive the ultrasonic signals sequentially, the at least one ultrasonic transducer being configured with a transmit phase for transmitting and a receive phase for receiving the returning ultrasonic signals, and wherein the at least one ultrasonic transducer is configured to receive the returning ultrasonic signals using time delays, summations, or both to focus the returning ultrasonic signals.

32. A method of monitoring a volume of a body cavity of a subject, wherein the volume of the body cavity varies over time, the method comprising:
emitting, by a transducer of a device attached to the subject, ultrasonic signals directed toward a target region that includes the body cavity, the emitted ultrasonic signals being emitted automatically at different times according to a timing schedule specified by the device;
receiving, by the transducer, reflected signals associated with the emitted ultrasonic signals reflected in the target region, each of the reflected signals corresponding to a respective emitted ultrasonic signal;
for each reflected signal of the reflected signals:
processing, by measurement circuitry of the device, the reflected signal to provide timing measurements of when reflected pulses are received;
determining, by the measurement circuitry, the volume of the body cavity using the timing measurements;
determining, by the measurement circuitry, whether the volume exceeds a threshold value; and
when the volume exceeds the threshold value for a given measurement, transmitting an alert signal from the measurement circuitry to alert circuitry, thereby causing an alert to be provided to the subject.

33. The method of claim 32, wherein the measurement circuitry includes one or more of an amplifier module, an analog to digital converter module, a digital multiplexer, a signal processing module, a measurement module, a monitoring module, an alarm module, a controller module, a communication module, and a memory.

34. The method of claim 32, wherein determining the volume exceeds the threshold value includes the volume increasing above the threshold value, the volume decreasing below the threshold value, or both.

35. The method of claim 32, further comprising determining, using the timing measurements, a distance between a first wall of the body cavity and a second wall of the body cavity along an emission line.

36. The method of claim 32, further comprising transmitting the timing measurements to a remote device configured to determine the volume of the body cavity.

37. (canceled)

38. The method of claim 32 further comprising detecting, by the measurement circuitry, a rate of change of the volume of the body cavity exceeds a rate of change threshold associated with a voiding of the body cavity.

39-48. (canceled)

49. A method comprising:
providing a transducer device comprising:
a first substrate;
a first electrode layer coupled to the first substrate;
a piezoelectric layer coupled to the first electrode layer;
a second electrode layer coupled to the piezoelectric layer;
a bonding oxide layer coupled to the second electrode layer;
providing a processing device comprising:
a device wafer;
one or more device wafer contacts coupled to the device wafer;
a device oxide layer coupled to the device wafer, the device oxide layer including one or more cavities;
bonding the bonding oxide layer to the device oxide layer;
after the bonding, removing the first substrate;
patterning a transducer area in the first electrode layer, the piezoelectric layer, and the second electrode layer;
creating a first via by etching the second electrode layer and the piezoelectric layer to expose the first electrode layer;
creating a second via by etching to expose the one or more device wafer contacts coupled; and
forming conductor channels in the first via and the second via.

50. The method of claim 49 further comprising patterning a plurality of transducer areas in the first electrode layer, the piezoelectric layer, and the second electrode layer.

51. The method of claim 49, wherein the device wafer includes processing electronics operable to supply a drive voltage to the transducer area.

52-54. (canceled)

55. The method of claim 49, wherein the device wafer includes processing electronics operable to measure a return voltage associated with the transducer area.

56-57. (canceled)

58. The method of claim 49, wherein providing the transducer device further comprises:
providing a handle wafer;
forming a first oxide layer coupled to the handle wafer;
forming a seed layer coupled to the first oxide layer;
forming the first electrode layer coupled to the seed layer;
forming the piezoelectric layer coupled to the first electrode layer;
forming the second electrode layer coupled to the piezoelectric layer; and
forming the bonding oxide layer coupled to the second electrode layer.

59. The method of claim 49, wherein providing the processing device further comprises:
providing the device wafer;
forming the device oxide layer coupled to the device wafer; and
forming a cavity in the device oxide layer.
